(19) United States
(12) Patent Application Publication
(10) Pub. No.: US 2019/0298911 A1
(43) Pub. Date: Oct. 3, 2019

(54) IV Set or IV Set System with Unidirectional Access Port

(71) Applicant: Somnus Medical, LLC, Roosevelt, UT (US)

(72) Inventors: Anthony Clark Harward, Roosevelt, UT (US); Lucas Reichert, Roosevelt, UT (US); Edwin T. Bulloch, Vernal, UT (US); Ronald E. Reichert, Washington, UT (US)

(21) Appl. No.: 16/292,214

(22) Filed: Mar. 4, 2019

Related U.S. Application Data
(60) Provisional application No. 62/637,999, filed on Mar. 2, 2018.

(51) Int. Cl. A61M 5/14; A61M 5/145; A61M 25/00; A61M 39/24

(52) U.S. Cl. CPC A61M 5/1408; A61M 5/1452; A61M 25/0097; A61M 2039/248; A61M 2039/2426; A61M 2039/2466; A61M 39/24

(57) ABSTRACT

A method for administering successive dosages of a medical fluid from a fluid injection device to a patient via an IV set during a medical procedure comprising: obtaining an IV set having a primary flow line defining a primary fluid path, and an access point defining an access port; establishing a fluid connection with a primary fluid in a fluid source with the primary fluid path; connecting a fluid injection device (e.g., syringe) to an unidirectional access port assembly comprising an anti-backflow device; injecting a first dosage of a secondary fluid (medication) from the fluid injection device into through an anti-backflow device (e.g., one-way check valve) and into the primary fluid path; maintaining the connection of the fluid injection device to the unidirectional access port assembly; and injecting a second dosage, or n number of additional dosages, of the secondary fluid through the anti-backflow device. Associated devices and systems are disclosed.

### RELATED APPLICATIONS

This application claims the benefit of U.S. Provisional Patent Application Ser. No. 62/637,999, filed Mar. 2, 2018, and entitled, “IV Set with Unidirectional Access Port,” which is incorporated by reference herein, in its entirety.

### BACKGROUND

One of the major benchmarks of medical care was the introduction of an Intravenous (IV) set to access the circulatory system of a patient, enabling the administration of fluids and medications in a controlled, predictable manner. The typical IV set includes a primary IV set having a primary fluid flow line of tubular construction with one or more access points. In some cases, a primary IV set can include a single IV line. In other cases, the primary IV set can include an IV set and an IV extension set attached thereto, these combining to form the primary IV set (and one type of an IV set system) and to define the primary flow line once connected together. Some of these access points can comprise access ports that allow the administration of medications, for example, through either a syringe by push or by infusion through another IV set (primary or secondary). A primary access point is located at one end of the IV set and is in communication with a fluid source, such as normal saline or some other carrier fluid, such as via a spike and drip chamber assembly positioned at a terminus or proximal end of the primary flow line. The primary IV set can further comprise, on a distal end, means for attachment to a patient injection site. Secondary IV sets defining secondary flow lines may be combined with the primary IV set and the primary flow line with similar construction options.

Use of IV sets has now become ubiquitous at every stage of medical care, including various types of conscious sedation procedures, such as endoscopic procedures. Conscious sedation procedures typically utilize a single IV set with one to three standard swab-able (able to be swabbed) connection sites, such as “Y”-sites. This IV set configuration is typically used because this is the only configuration that IV set suppliers currently provide. An access point close to the patient catheter site is generally the connection location for syringe(s) containing the drug used to sedate the patient. Occasionally, a secondary IV set is attached to the upper “Y”-site through which antibiotic or other supplemental fluid is added to the primary IV line.

During the procedure, the syringe is removed from the standard “Y”-site, for instance, after each of multiple drug doses to prevent carrier fluid (which is under pressure) in the IV set from entering the syringe by pushing the plunger backwards and diluting the drug within the attached syringe (also known as “backflow” or “reflux”). If dilution of the drug within the syringe should occur, the clinician will no longer have definitive knowledge or control of the amount or potency of the drug being administered to the patient during subsequent “pushes” or dosages.

Furthermore, each time the drug filled syringe is removed from a connection site, such as a “Y”-site, the IV system becomes “open,” thus exposing the patient to infection risks. Additionally, the “open” drug syringe may be placed on a non-sterile surface near the patient, until the next dose is required. This is time consuming and a dangerous practice as drug dosing typically occurs multiple times throughout the duration of the procedure. Each time the syringe is removed from its connection site, thus creating an “open” system, this exposes the patient to potential infection risks. Efforts to try and mitigate such risks involve the clinician covering the swab-able port, or swabbing the port with a disinfectant between each attachment and re-attachment of the drug syringe to disinfect the port. Unfortunately, proper coverage or swabbing of the attachment port is often neglected during conscious sedation procedures.

### BRIEF DESCRIPTION OF THE DRAWINGS

Features and advantages of the invention will be apparent from the detailed description which follows, taken in conjunction with the accompanying drawings, which together illustrate, by way of example, features of the invention; and wherein:

FIG. 1A is an isometric view of an IV extension set comprising a unidirectional access port assembly in accordance with an example of the present disclosure;

FIG. 1B is a plan view of an IV fluid delivery system comprising the IV extension set with the unidirectional access port assembly of FIG. 1A, in accordance with an example of the present disclosure;

FIG. 1C illustrates a plan view of an IV fluid delivery system comprising a unidirectional access port assembly in accordance with an example of the present disclosure;

FIG. 2A illustrates a cross-sectional view of a unidirectional access port assembly usable with the IV fluid delivery systems of FIGS. 1B and 1C, and in a closed position and coupled to a fluid injection device, in accordance with an example of the present disclosure;

FIG. 2B illustrates the unidirectional access port assembly FIG. 2A, in an open position;

FIG. 3A illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure;

FIG. 3B illustrates the unidirectional access port assembly FIG. 3A in an open position:

FIG. 4A illustrates a cross-sectional view of a unidirectional access port assembly usable with the IV fluid delivery system of FIGS. 1B and 1C, the unidirectional access port assembly shown as being in a closed position and coupled to a fluid injection device, in accordance with an example of the present disclosure;

FIG. 4B illustrates the unidirectional access port assembly of FIG. 4A in an open position;

FIG. 5 illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure;

FIG. 6 illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure;

FIG. 7 illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure;

FIG. 8 illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure;

FIG. 9 illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure:

FIG. 10 illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure;

FIG. 11A illustrates a cross-sectional view of a unidirectional access port assembly usable with the IV fluid delivery systems of FIGS. 1B and 1C, the unidirectional access port assembly shown as being in a closed position and coupled to a fluid injection device, in accordance with an example of the present disclosure; and

FIG. 11B illustrates the unidirectional access port assembly of FIG. 11A in an open position.

### DETAILED DESCRIPTION

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein. “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

An initial overview of technology examples is provided below and then specific technology examples are described in summary and in further detail later. This initial summary is intended to aid readers in understanding the technology more quickly, but is not intended to identify key features or essential features of the technology, nor is it intended to limit the scope of the subject matter.

At the outset, an “IV set” is intended to mean a single IV line. An “IV set system” is intended to describe an IV set connected with at least one additional IV set (e.g., a secondary IV set) or an IV extension set. An “IV fluid delivery system,” is intended to mean at least one IV set or IV set system operable with one or more other components, such as a fluid chamber (e.g., gravity bag type fluid delivery source, pump type fluid delivery source), a patient interface device (e.g., a catheter), or others for delivery of a fluid to a patient intravenously during a medical procedure, such as an endoscopic procedure, an extension set, or any combination of these. An “access point” is intended to mean any point along an IV set or IV set system in which access is provided to the fluid flow within the IV set or IV set system. An access point can include such elements as access ports, spike/drip chambers, patient interconnect structures, fluid interconnection means, and others as will be apparent to those skilled in the art. An “access port” is intended to mean a specific type of access point that facilitates access to the fluid flow within the IV set or IV set system, such as by push (e.g., using a syringe), by infusion (e.g., through coupling of another IV set, a manifold, an extension set, or others).

The present disclosure sets forth a method for administering successive dosages of a medical fluid (e.g., anesthesia drug) from a fluid injection device to a patient via an IV set during a conscious sedation (e.g., endoscopic) or other medical procedure. The method can comprise: obtaining an IV set having a primary flow line defining a primary fluid path, and a plurality of access points, at least one of the plurality of access points defining an access port, establishing a fluid connection with a primary fluid in a fluid source, such as a gravity-based IV solution bag, with the primary fluid path of the IV set through one of the plurality of access points; connecting a fluid injection device to a unidirectional access port assembly to access the access port, the access port facilitating fluid communication between the fluid injection device and the primary fluid path through an anti-backflow device of the unidirectional access port assembly, the fluid injection device comprising a secondary fluid (e.g., an anesthesia drug); injecting a first dosage of the secondary fluid from the fluid injection device into the access port and the primary fluid flow path through the anti-backflow device; maintaining the connection of the fluid injection device to the unidirectional access port assembly; and injecting a second dosage (or any number (n number) of additional dosages) of the secondary fluid from the fluid injection device into the access port and the primary fluid flow path through the anti-backflow device.

In one example, the method further comprises maintaining the connection of the fluid injection device to the unidirectional access port assembly, and injecting a third dosage of the secondary fluid from the fluid injection device into the access port and the primary fluid flow path through the anti-backflow device.

In one example, the anti-backflow device comprises a check valve operable to prevent the primary fluid from passing through the check valve into the fluid injection device between injection of the first dose and injection of the second dose.

In one example, the operation of connecting the fluid injection device to the unidirectional access port assembly to access the access port comprises connecting a syringe to the unidirectional access port assembly by engaging (e.g., threadably) a female type connection portion (e.g., female Luer lock) of the syringe to an external male type connection portion (e.g., male Luer lock) of the unidirectional access port assembly. By doing so, the internal male portion of the syringe is engaged with the internal female portion of the unidirectional access port assembly.

In one example, the operation of maintaining the connection of the fluid injection device to the unidirectional access port assembly comprises facilitating prevention of backflow of the primary fluid into the secondary fluid injection device via the anti-backflow device comprising a check valve, thereby preventing dilution of the secondary fluid with the primary fluid.

The present disclosure sets forth a method for preventing dilution of a medical fluid in a fluid injection device coupled to an IV set during successive dosages with the fluid injection device. The method comprises: obtaining an IV set having a primary flow line defining a primary fluid path, and a plurality of access points, at least one of the plurality of access points defining an access port as part of a unidirectional access port assembly; establishing a fluid connection with a primary fluid in a fluid source, such as a gravity-based IV solution bag, with the primary fluid path of the IV set through one of the plurality of access points; connecting a fluid injection device to the access port of the unidirectional access port assembly to access the access port, the access port facilitating fluid communication between the fluid injection device and the primary fluid path through an anti-backflow device of the unidirectional access port assembly, the fluid injection device comprising a secondary fluid (e.g., an anesthesia drug); applying pressure to the fluid injection device to inject the secondary fluid from the fluid injection device into the primary fluid path through the anti-backflow device; and removing pressure from the fluid injection device, while maintaining connection of the fluid injection device to the unidirectional access port assembly, in a manner such that the anti-backflow device restricts fluid flow from the primary fluid path to the fluid injection device, thereby preventing dilution of the secondary fluid in the fluid injection device.

The present disclosure sets forth a method for manufacturing an IV set (e.g., an endoscopic IV set, or an IV set for conscious sedation medical procedures) comprising: forming a primary flow body comprising a sidewall and opposing first and second openings, and a flow channel extending between the first and second openings; forming an access port body supported at least partially by the primary flow body and that defines an access port operable to receive a fluid injection device; coupling an anti-backflow device to at least one of the primary flow body or the access port body, wherein the primary flow body, the access port body, and the anti-backflow device comprise a unidirectional access port assembly; and coupling the primary flow body of the unidirectional access port assembly to at least a portion of a primary flow line of an IV set that defines a primary fluid path, wherein the unidirectional access port assembly is operable to maintain continuous connection to the fluid injection device over multiple successive dosages of a secondary fluid (e.g., an anesthesia drug) from the fluid injection device into a primary fluid about the primary fluid path of the IV set through the anti-backflow device.

The present disclosure sets forth an IV set comprising: a plurality of primary flow line segments defining, at least in part, a primary flow line and a primary fluid path, the IV set comprising a plurality of access points, at least one of the plurality of access points being an access port; and a unidirectional access port assembly at least partially defining the primary flow line and the primary fluid path. The access port assembly comprises: a primary flow body comprising a sidewall and opposing first and second openings, and a flow channel extending between the first and second openings, the primary flow body being coupled to at least one of the primary flow line segments; an access port body supported at least partially by the primary flow body and that defines an access port operable to receive a fluid injection device; and an anti-backflow device supported at least partially by the access port body, and facilitating fluid communication to the primary fluid path via the access port. The unidirectional access port assembly being operable to maintain continuous connection to a fluid injection device over multiple successive dosages of a fluid from the fluid injection device into the primary fluid path through the anti-backflow device.

In one example, the primary flow body and the access port body at least partially define a T-port type access port.

In one example, the primary flow body and the access port body at least partially define a Y-site type access port.

In one example, the anti-backflow device comprises an elastomeric component that fluidly separates the access port from the flow channel.

In one example, the access port body comprises a male type connection portion (e.g., Luer lock) configured to removably couple a female type connector portion (e.g., Luer lock) of the fluid injection device.

The present disclosure sets forth an IV fluid delivery system for a patient during an endoscopic or other type of conscious sedation medical procedure comprising: a fluid source, such as a gravity-based IV solution bag, containing a primary fluid, and an IV set comprising: a plurality of primary flow line segments defining, at least in part, a primary flow line and a primary fluid path for transferring the primary fluid from the fluid source, the IV set comprising a plurality of access points, at least one of the plurality of access points being an access port; a fluid injection device comprising a secondary fluid (e.g., an anesthesia drug); and a unidirectional access port assembly at least partially defining the primary flow line and the primary fluid path. The access port assembly comprises: a primary flow body comprising a sidewall and opposing first and second openings, and a flow channel extending between the first and second openings, the primary flow body being coupled to at least one of the primary flow line segments; an access port body supported at least partially by the primary flow body and that defines an access port that receives the fluid injection device; and an anti-backflow device supported at least partially by the access port body, and facilitating fluid communication to the primary fluid path via the access port. The unidirectional access port assembly is operable to maintain continuous connection to the fluid injection device over multiple successive dosages of the secondary fluid from the fluid injection device into the primary fluid path through the anti-backflow device.

In one example, the primary flow body and the access port body at least partially define one of a T-port type access port or a Y-site type access port.

In one example, the anti-backflow device comprises one of an elastomeric component or a ball type check valve, and is operable to prevent a primary fluid from passing through the anti-backflow device into a fluid chamber of the fluid injection device while the fluid injection device is removably coupled to the access port body.

The present disclosure sets forth an intravenous (IV) extension set for use with another IV set, such as a primary or standard IV set. The IV extension set can comprise: a plurality of extension flow line segments defining, at least in part, an extension flow line and an extension fluid path; a first connector portion coupled to one of the extension flow line segments (the first connector portion configured to couple to a connector portion of an IV set (e.g., a standard IV set, such as a gravity IV set); a second connector portion coupled to another one of the extension flow line segments (the second connector portion configured to couple to a patient interface device, such as a catheter); and a unidirectional access port assembly coupled to adjacent extension flow line segments. The unidirectional access port assembly can comprise: a primary flow body comprising a sidewall and opposing first and second openings, and a flow channel extending between the first and second openings; an access port body supported at least partially by the primary flow body and that defines an access port operable to receive a fluid injection device; and an anti-backflow device supported at least partially by the access port body, and facilitating fluid communication to the extension fluid path via the access port body.

The unidirectional access port assembly can be operable to maintain continuous connection to a fluid injection device over multiple successive dosages of a fluid from the fluid injection device into the primary fluid path through the anti-backflow device.

The present disclosure still further sets forth a method for facilitating the administration of successive dosages of a secondary fluid from a fluid injection device to a patient during a medical procedure via an IV fluid delivery system, the method comprising obtaining an IV set or an IV set system (e.g., an IV set connected to an IV extension set) operable to receive fluid from a fluid source, and defining a primary fluid flow line and a potential primary fluid flow path, the IV set or IV set system having a unidirectional access port assembly associated with an access port of the IV set or IV set system. The method can further comprise connecting a fluid injection device to the unidirectional access port assembly to access the access port, the access port facilitating fluid communication between the fluid injection device and the primary fluid flow path through an anti-backflow device of the unidirectional access port assembly, the fluid injection device being capable and configured to receive and dispense a secondary fluid into the primary fluid flow line and the potential primary fluid flow path (once the IV fluid delivery system is put into use). The fluid injection device can form a part of the IV fluid delivery system.

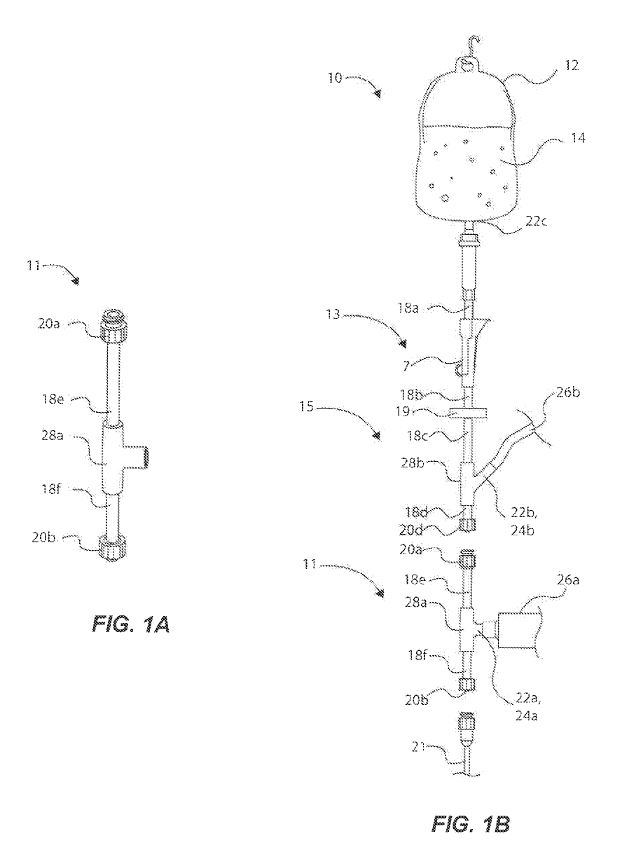

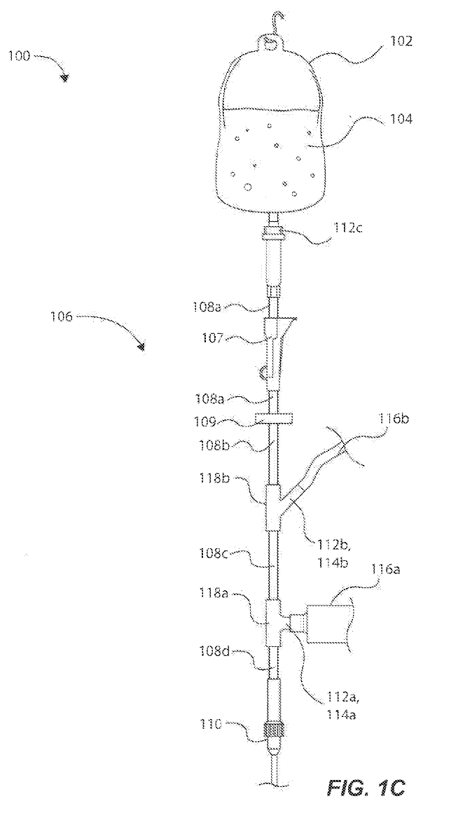

FIG. 1A illustrates an IV extension set 11, and FIG. 1B illustrates an IV fluid delivery system 10 incorporating the IV extension set 11, in accordance with an example of the present disclosure, wherein the IV fluid delivery system 10 is operable for use during a conscious sedation medical procedure, such as an endoscope medical procedure. As an overview, the IV extension set 11 can be coupled to an IV set 13 as an extension of the IV set 13 to form an IV set system operable within the IV fluid delivery system 10. For example, the IV set 13 can comprise an existing or standard, readily available IV set that is configured to receive and connect to the IV extension set 11 to facilitate fluid delivery to a patient during a particular identified medical procedure. It is noted herein that, depending upon the desired intended use, the IV set 13 can function by itself as a standalone an IV set, such as a primary IV set defining a primary flow line, as a secondary IV set defining a secondary flow line, or any other IV set as will be recognized by those skilled in the art. In another aspect, the IV set 13 can be connected to the IV extension set 11, and these together can define and operate as an IV set system, which can also be considered as a primary IV set (these together establishing a primary flow line), such as the primary IV set 15 of the IV fluid delivery system 10 shown in FIG. 1B. As used herein, the term “primary IV set” is intended to cover and include a single, standalone IV set (e.g., a single IV line) that establishes and defines the primary flow line, as well as an IV set connected with an IV extension set, wherein the IV set and the extension set operate together to establish and define the primary flow line. Each of these scenarios facilitates establishment of a primary flow through the primary flow line.

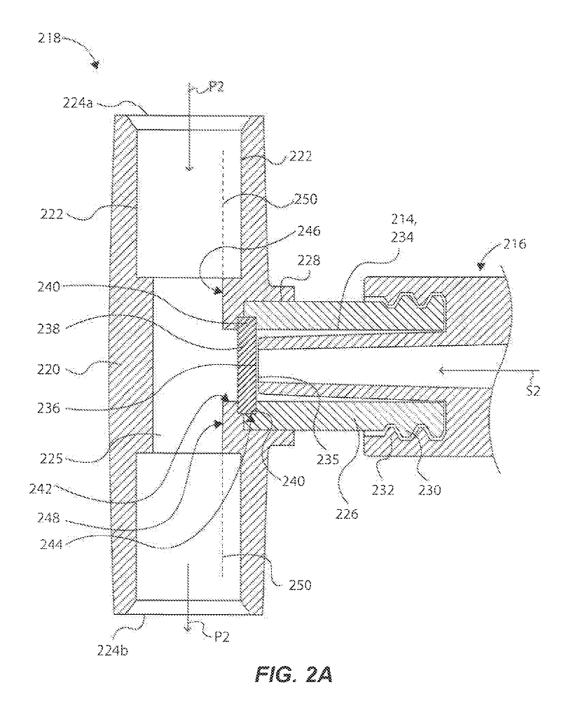

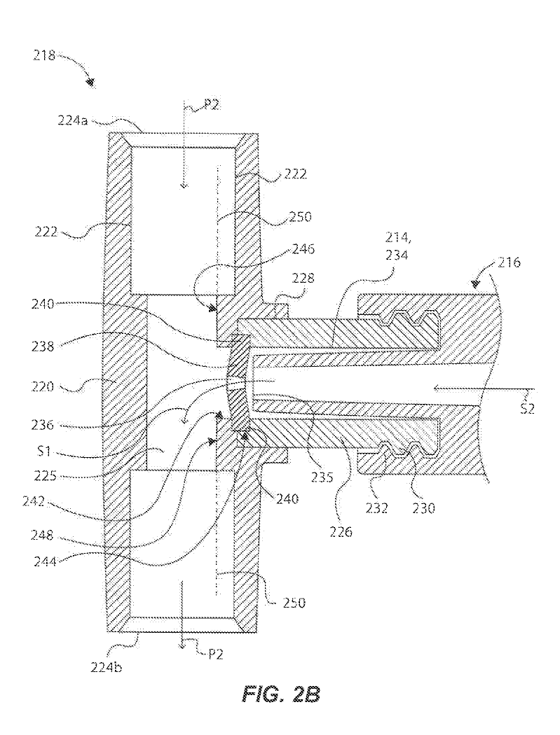

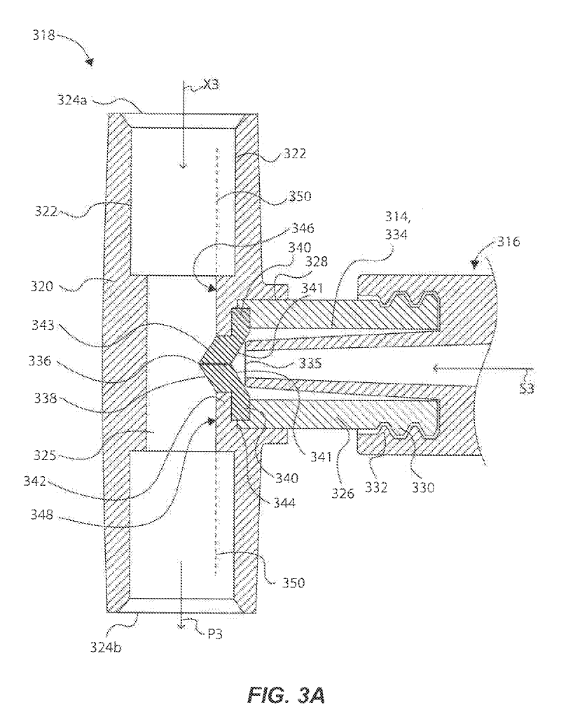

As indicated above, the IV fluid delivery system 10 can comprise the IV set 13 and the IV extension set 11, these operating together to define the primary IV set 15. The IV extension set 11 can function in a similar manner as prior extension sets, namely to connect to an existing IV set. However, unlike prior IV extension sets, the IV extension set 11 discussed herein can provide added functionality to a primary IV set, as described in more detail below. In the example shown, the IV extension set 11 can comprise first and second extension flow line segments 18e and 18f defining, at least in part, an extension flow line and an extension fluid flow path that extends through the IV extension set 11. As discussed above, upon connection of the extension set 11 with an IV set, such as IV set 13, the extension flow line operates as part of the primary flow line, and the extension fluid flow path operates as part of the primary flow path. The IV extension set 11 can comprise a first connector portion 20a (e.g., a male or female Luer lock, or other type of connector or connector portion), which can be coupled to or otherwise associated with an end of the first extension flow line segment 18e. The IV extension set 11 can further comprise a second connector portion 20b (e.g., a male or female Luer lock, or other type of connector or connector portion), which can be coupled to or otherwise associated with an end of the second extension flow line segment 18f. The IV extension set 11 can further comprise a unidirectional access port assembly 28a coupled to the adjacent first and second extension flow line segments 18a and 18b, such that the unidirectional access port assembly 28a is supported about the line segments 18a and 18b, and is situated between the ends of the IV extension set and between the first and second connector portions 20a and 20b. Although the unidirectional access port assembly 28a is shown as being in the form of a T-port type access port, it can comprise any one of the example unidirectional access port assemblies of FIGS. 2-11B.

As shown in FIG. 1B, the IV fluid delivery system 10 can further comprise a fluid source (e.g., see gravity-based IV solution bag 12) containing a primary fluid 14 (e.g., saline, medication, or others as will be known to those skilled in the art), and the IV set 13 coupled to the fluid source (the gravity-based IV solution bag 12) (e.g., via an access point (e.g., see access point 22c in the form of a spiked drip chamber). In another example, although not shown, instead of a gravity-based IV solution bag, the IV fluid delivery system 10 can comprise a pump or pump set operable with the IV set 13, and operable to pump fluid through the IV set 13.

The IV set 13 can comprise a plurality of primary flow line segments 18a-d defining, at least in part, a primary flow line and a primary fluid path for facilitating the transfer of the primary fluid 14 from the fluid source (e.g., gravity-based IV solution bag 12 (or a fluid pump)) to a patient (e.g., via a catheter 21).

The primary IV set 15 comprises a plurality of access points 22a-c. Access point drip chamber 22c can be a spike/drip chamber assembly for introduction of the primary fluid from the solution bag 12 to the primary fluid line. Access points 22a and 22b can comprise access ports 24a and 24b for introduction of a secondary fluid (e.g., an anesthesia drug or other type of fluid) into the primary fluid for delivery to the patient. More specifically, a fluid injection device 26a, such as a needleless syringe containing the secondary fluid, such as a medication (e.g., anesthesia drug) can be coupled to the access port 24a as an injection site. In one example, the access port 24a can be defined by the unidirectional access port assembly 28a, and the access port 24b can be defined by a bi-directional access port assembly 28b. Fluid infusion device 26b, such as a secondary IV set defining a secondary fluid line containing a secondary fluid (e.g., anesthesia drug), can be removably coupled to the bi-directional access port assembly 28b proximate the access port 24b. A roller clamp 7 can be positioned over, and operable with, line segment 18a to control the rate of flow of the primary fluid through the IV set 13. An in-line check valve 19 can be permanently bonded to the flow line segment 18a to prevent backflow into the solution bag 12.

As noted above, the IV extension set 11 can be removably coupled to the IV set 13 to extend and to form the primary IV set 15 by coupling the connector portion 20a on the IV extension set 11 to a mating connector portion 20d on the IV set 13, wherein the first extension flow line segment 18e of the IV extension set 11 is joined to the flow line segment 18d of the IV set 13, these being fluidly coupled to one another (meaning fluid is able to flow between them) so as to define, at least in part, the primary flow line, and to establish, at least in part, the primary flow path once the primary IV set 15 is put into use. Likewise, the connector portion 20b on the IV extension set 11 can be connected to a connection portion of a patient interface device, such as a catheter (e.g., see the catheter 21), thus fluidly coupling the primary IV set 15 (or in other words the extended primary IV set 15) to the catheter 21. The IV set 13 and IV extension 11 set can be connected to each other and primed prior to connection to the patient catheter.

A method or operation of administering successive dosages as discussed below regarding FIG. 1C can be achieved via the IV system 10 of FIG. 1B, such as delivering successive dosages of secondary fluid from the syringe 26a through the unidirectional access port assembly 28a into the extended fluid flow path of the IV extension set 11 (which extended fluid flow path defines, at least in part, the primary fluid flow path) for mixing with the fluid 14 of the primary IV set 15.

In the example shown, the unidirectional access port assembly 28a is supported on and carried by the IV extension set 11 at a midpoint location. In addition, only a single unidirectional access port assembly 28a is shown. However, in other examples, a plurality of unidirectional access port assemblies can be supported on or included in an IV extension set. Moreover, the one or more unidirectional access port assemblies can be located anywhere along the IV extension set.

Unlike prior extension sets, the IV extension set 11 discussed herein can comprise unidirectional access port functionality and capabilities. Therefore, once the IV extension set 11 is connected to an other IV set (e.g., a standard gravity, pump or other IV set or IV line), the resulting IV system becomes an IV set or IV set system having a unidirectional access port, thus providing the entire IV set system (namely the primary IV set as defined by the IV set and the IV extension set combination) with unidirectional access port functionality and capabilities. Indeed, the resulting primary IV set or IV set system provides a unidirectional access port to which a syringe (or needleless connector (e.g., needleless Luer connector) can be directly connected. The unidirectional access port functionality, capabilities and advantages are described in greater detail below, but essentially this valve functions to permit a syringe to remain attached to an IV Set while preventing fluid to flow from the IV set back into the syringe (i.e., preventing backflow). Again, it is contemplated that an IV extension set, such as the IV extension set 11 discussed herein, can comprise any of the example unidirectional access port assemblies discussed below. However, these are not intended to be limiting in any way, as will be appreciated by those skilled in the art.

FIG. 1C illustrates an IV fluid delivery system 100 for facilitating the delivery of a fluid to a patient during a conscious sedation medical procedure, such as an endoscope medical procedure, in accordance with an example of the present disclosure. The system 100 can comprise a fluid source, such as a gravity-based IV solution bag 102, containing a primary fluid 104 (e.g., saline), and an IV set 106 coupled to the fluid source (gravity-based IV solution bag 102) (e.g., via an access point (e.g., see access point 112c in the form of a drip chamber)). The IV set 106 can comprise a plurality of primary flow line segments 108a-d defining, at least in part, a primary flow line through which a primary fluid path can be established for transferring the primary fluid 104 from the gravity-based IV solution bag 102 to a patient (e.g., via a catheter 110 of the IV set 106) upon use of the IV fluid delivery system 100. The catheter 110 can be coupled to line segment 108d via a male connection, such as a male Luer lock. A roller clamp 107 can be positioned over, and operable with, line segment 108a to control the rate of flow of the primary fluid through the IV set 106. The IV set 106 further comprises a plurality of access points 112a-c. Access point drip chamber 112c is a spike/drip chamber assembly for introduction of the primary fluid from the solution bag 102 to the primary fluid line. Access points 112a and 112b can comprise access ports 114a and 114b for introduction of a secondary fluid (e.g., an anesthesia drug or other type of fluid) into the primary fluid for delivery to the patient. More specifically, a fluid injection device 116a, such as a needleless syringe containing the secondary fluid, such as a medication (e.g., anesthesia drug) can be coupled to the access port 114a as an injection site. In one example, the access port 114a can be defined by a unidirectional access port assembly 118a, which can be shaped similar to a bondable T-port type connector, and configured such as any one of the unidirectional access port assemblies described in the present disclosure having a T-port type configuration. As will be discussed in more detail below, the unidirectional access port assembly 118a can provide one-way fluid flow, or in other words, can prevent backflow of fluid into the fluid injection device 116a. The access port 114b can be defined by a bi-directional access port assembly 118b. Fluid infusion device 116b, such as a secondary fluid line containing a secondary fluid (e.g., anesthesia drug), can be removably coupled to the bi-directional access port assembly 118b proximate the access port 114b. An in-line check valve 109 can be permanently bonded to one of the flow line segments to prevent backflow into the fluid source (e.g., the solution bag 102).

As an overview, and in one example of a method for administering successive dosages of a medical fluid from the fluid injection device 116a to a patient via the IV set 106, once a clinician has established a fluid connection to the patient intravenously with the primary fluid 104 via the primary fluid flow path of the IV set 106, the clinician can connect the fluid injection device 116a to the unidirectional access port assembly 118a to access the access port 114a, and to introduce one or more dosages of a fluid within the fluid injection device 116a into the primary fluid flow path to mix with the primary fluid 104. The access port 114a facilitates fluid communication between the fluid injection device 116a and the primary fluid flow path through an anti-backflow device (e.g., a one-way check valve) of the unidirectional access port assembly 118a. Examples of “anti-backflow devices” will be further detailed below, as having one-way check valves that are self-sealing to prevent backflow of the primary fluid into the fluid injection device. Advantageously, the clinician can inject a first dosage of the secondary fluid from the fluid injection device 116a into the access port 114a through the anti-backflow device of the unidirectional access port assembly 118a and into the primary fluid path to mix with the primary fluid 104 prior to delivery to the patient. As a further advantage, the clinician can continuously maintain the connection of the fluid injection device 116a to the unidirectional access port assembly 118a prior to injecting a second dosage, thus permitting the clinician to inject the second dosage of the secondary fluid from the fluid injection device 116a into the access port 114a through the anti-backflow device of the unidirectional access port assembly 118a and into the primary fluid path for mixing with the primary fluid 104, all while maintaining the connection of the fluid injection device 116a to the unidirectional access port assembly 18a. This can be repeated for any number of successive dosages until the fluid injection device is empty. Advantageously, repeated attachment, detachment and reattachment of the fluid injection device 116a (e.g., a drug syringe) to the IV set is avoided, as are the attendant risks and costs associated with this practice.

Because of the configuration of the unidirectional access port assembly 118a having an anti-backflow device, the primary fluid cannot reflux or “flow back” into the fluid injection device 116a, which would cause dilution of the secondary fluid and an increase in volume of fluid within the fluid injection device 116a, which could be problematic to the patient because the clinician may be unaware of the actual amount of secondary fluid (e.g., anesthesia drug) that has been delivered to the patient, because the fluid injection device 116a could potentially have a certain amount of primary fluid therein, thus the fluid injection device 116a could potentially indicate a certain volume of “presumed” secondary fluid therein, when in fact, there may also be an amount of primary fluid that has back flowed into the fluid injection device 116a). As discussed above, this can be quite undesirable and can cause health issues with the patient, such as those associated with incorrect dosages of the secondary fluid. On the other hand, with the present technology, the clinician can maintain continuous connection of the fluid injection device 116a to the unidirectional access port assembly 118a without fear of the primary fluid back flowing into the fluid injection device 116a.

By having the capability of maintaining the connection of the fluid injection device 116a to the unidirectional access port assembly 118a in this manner, the clinician does not need to remove the fluid injection device 116a between successive dosages of the secondary fluid to the patient, which would otherwise (with prior connections without anti-back flow functionality) normally necessitate sterilizing (e.g., swabbing) the access port 112a of the port, and also sterilizing the tip of the removed fluid injection device 116a. Thus, sterilization at this access port 112a is no longer required between successive dosages from a given syringe because of the configuration of the unidirectional access port assembly 118a, which dramatically reduces or eliminates the possibility of contamination of the access port 112a and infection to the patient from this access port 112a.

Detailed examples of different unidirectional access port assemblies capable of performing the aforementioned functionality are set forth below.

It is noted that while the unidirectional access port assemblies can be associated with a primary IV set or primary IV set system, it is contemplated that the unidirectional access port assemblies can be associated with any type of IV set for any desired or needed purpose, where unidirectional flow or anti-backflow functionality is desired or needed. As such, references to a primary IV set are not intended to be limiting in any way.

FIG. 2A illustrates an example unidirectional access port assembly 218 in a closed position and coupled to a fluid injection device 216, and FIG. 2B illustrates the unidirectional access port assembly 218 in an open position. The unidirectional access port assembly 218 comprises a primary flow body 220 comprising a sidewall 222 and opposing first and second openings 224a and 224b. A flow channel 225 extends between the first and second openings 224a and 224b. The primary flow body 220 can be coupled to respective flow line segments (e.g., extension flow line segments 18e and 18f of FIGS. 1A and 1B, or primary flow line segments 108c and 108d of FIG. 1C) via respective first and second openings 224a and 224b, such as by a medical grade adhesive. Thus, the unidirectional access port assembly 218 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The unidirectional access port assembly 218 can comprise an access port body 226 supported at least partially by the primary flow body 220. The access port body 226 may be formed integral with the primary flow body 220, or it may be a separate component (as shown) that is attached to the primary flow body 220. In one example, the access port body 226 can extend outwardly and orthogonally from the sidewall 222 to form generally a T-shape configuration. In another example, the access port body 226 can comprise a Y-shape configuration. The access port body 226 can be generally cylindrically shaped having one end attached (e.g., glued, ultrasonically welded, or press fit) into a side opening 228 of the primary flow body 220. On the other end, the access port body 226 can comprise an access connector interface that facilitates the removable coupling or connection of a fluid injection device, such as a syringe (i.e., that interfaces with and that engages and removably couples the fluid injection device). In one example, the access port body 226 can comprises a male type connection portion 230 configured to removably couple a female type connector portion 232 of a fluid injection device 216, such as a male-female Luer lock configuration. The access port body 226 further comprises an access port opening 234 that defines an access port 214, and that receives an injection end 235 of the fluid injection device 216 for delivery of a secondary fluid from a fluid chamber (not shown) of the fluid injection device 216 into the access port 214 via a secondary fluid path S2 of the fluid injection device 216. Although not shown here, a split septum device could be supported within the access port opening 234 through which the injection end 235 can extend through for sealing the injection end 235 to the access port body 226. Split septum devices could also be incorporated into the other access port bodies discussed in the examples herein, and as shown in the various figures. The access port opening 234 can also be a female Luer which interfaces directly with the male Luer of the injection device 216.

The unidirectional access port assembly 218 can further comprise an anti-backflow device 238 supported at least partially by the access port body 226, and that facilitates fluid communication to a primary fluid path P2 (of an IV set) via the access port 214. More specifically, the anti-backflow device 238 can comprise an elastomeric component that fluidly separates the access port 214 from the flow channel 225, and which can comprise a one-way compliant opening 236 operable to prevent the primary fluid from passing through the one-way compliant opening 236 into the fluid chamber of the fluid injection device 216 while the fluid injection device 216 is removably coupled to the access port body 226. In this example, the anti-backflow device 238 can be similar to a “duck-bill” type of elastomeric check valve, such that the compliant opening 236 is a slit formed through the anti-backflow device 238 (as opposed to a true aperture formed by removing material from the anti-backflow device 238 so that fluid can pass through). Thus, in the closed position of FIG. 2A, the one-way compliant opening 236 prevents or restricts the primary fluid from passing into the access port opening 234 because the elastomeric properties of the anti-backflow device 238 are such that the anti-backflow device 238 is rigid enough to overcome the fluid pressure applied by the primary fluid in the primary fluid path P1 to remain closed, and because the compliant opening 236 is formed as a slit so that the primary fluid cannot pass through without sufficient fluid pressure. In the open position of FIG. 2B, the one-way compliant opening 236 permits or allows the secondary fluid to pass from the fluid injection device 216 through the access port opening 234 and into the primary fluid path P2, because the elastomeric properties of the anti-backflow device 238 are such that the anti-backflow device 238 is compliant enough to displace or open as a result of the fluid pressure of the secondary fluid applied by the fluid injection device 216 on one side of the anti-backflow device 238 such that the slit opens up and protrudes inwardly towards the primary fluid path for mixing the secondary fluid with the primary fluid. In one example, the anti-backflow device 238 can be a planar disk in the shape of a cylinder, but it can take other suitable shapes and forms.

The anti-backflow device 238 can be supported by the access port body 226 and/or the primary flow body 220 in a number of ways. For instance, a perimeter portion 240 of the anti-backflow device 238 can be supported by and attached to an annular recess formed in only the access port body 226 or an annular recess of only the primary flow body 220, such that the perimeter portion 240 of the anti-backflow device 238 is fixed, and so that the central area of the anti-backflow device 238 can flex or stretch about the one-way compliant opening 236.

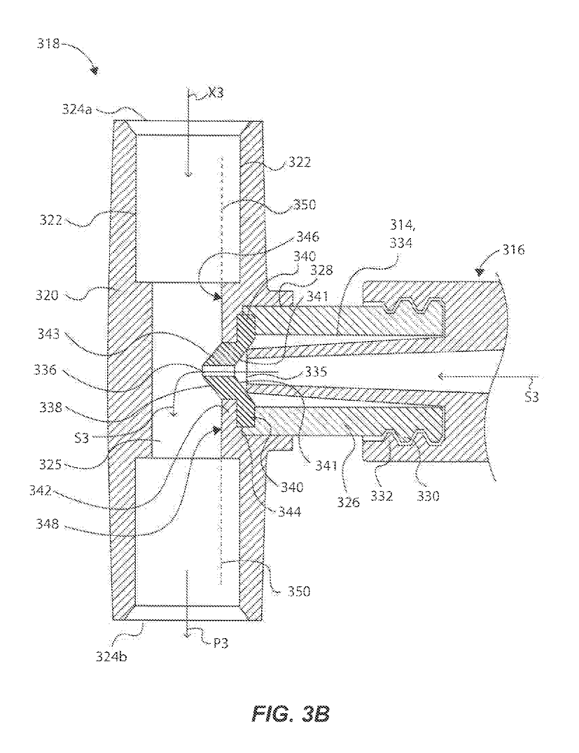

In the example shown in FIGS. 2A and 2B, the primary flow body 220 and the access port body 226 cooperate to retain and support the perimeter portion 240 of the anti-backflow device 238. More specifically, the primary flow body 220 can comprise a shoulder portion 242 that supports a corner portion of the perimeter portion 240 of the anti-backflow device 238, and the access port body 226 can comprise a complimentary shoulder portion 244 that supports an opposing corner portion of the perimeter portion 240. Thus, the perimeter portion 240 of the anti-backflow device 238 can be somewhat sandwiched or press fit between respective shoulder portions 242 and 244 to axially and radially support the anti-backflow device 238. A medical grade adhesive may be applied between the perimeter portion 240 and the shoulder portions 242 and 244 to fix or attach the anti-backflow device 238 in-place. Thus, the anti-backflow device 238 fluidly separates the access port 214 from the flow channel 225 when in the closed position, as in FIG. 2A. And, the anti-backflow device 238 is “integrated” within the structure defined by the primary flow body 220 and the access port body 226 (as is the case with the anti-backflow devices described regarding FIGS. 3A-8). This promotes sterility and provides simplicity of the system, thereby reducing the risk of component failure while reducing risk of infection to the patient.

Notably, the injection end 235 of the fluid injection device 216 can be positioned very near one side of the anti-backflow device 238, such as 1 to 2 millimeters, to account for tolerances between the fluid injection device 216 and the access port body 226. Thus, any possible “dead-space” is minimized because the injection end 235 is juxtaposed with the anti-backflow device 238. Ideally, the injection end 235 would slightly touch the side surface of the anti-backflow device 238 when in the closed position, thereby entirely eliminating any “dead-space” between the fluid injection device 216 and the anti-backflow device 238. Dead-space can be considered any void or space between the injection end 235 of the fluid injection device 216 and the primary fluid flow path that, if present, could have the potential to collect fluid, such as air or secondary fluid. In other words, dead space can be considered a “no fluid space.” In existing systems where such dead-space exists, contamination or dilution of the secondary fluid can occur, or air bubbles may exist in this dead space. Too much dead-space can even cause some amount of secondary fluid to be trapped, thereby never reaching the patient. Reducing or eliminating dead-space in this manner of the present disclosure helps to eliminate or reduce the amount of medication (secondary fluid) needed because the medication will not be trapped in the dead-space; rather, it will be appropriately transferred to the patient. This also reduces or eliminates the time needed to purge air from the unidirectional access port assembly that may be trapped in such dead-space, which air can be detrimental to patient if injected intravenously.

On the other side of the anti-backflow device 238 adjacent the primary fluid path P2, the anti-backflow device 238 can be positioned proximate or near the primary fluid path P2, thereby reducing or minimizing dead-space between the primary flow path and the anti-backflow device 238. More specifically, the sidewall 222 of the primary flow body 220 can comprise an upper planar sidewall portion 246 and a lower planar sidewall portion 248 that define a flow plane 250 along which the primary fluid generally flows through the flow channel 225. When in the open position of FIG. 2B, the one-way compliant opening 236 extends inwardly toward this flow plane 250, so that the secondary fluid can be injected/mixed with the primary fluid at a mixing region that is generally within the primary fluid path P2. Thus, any possible dead-space is minimized or eliminated because of the lateral position of the one-way compliant opening 236 being adjacent the primary fluid path P2.

As alluded to above, the unidirectional access port assembly 218 is operable to maintain continuous connection of the fluid injection device 216 with the access port 226 over multiple successive dosages of the secondary fluid from the fluid injection device 216 into the primary fluid path P2 through the anti-backflow device 238. This is because when pressure is removed from a plunger (or other movable device) of the fluid injection device 216 (such as after a first dosage is injected, as in FIG. 2B), fluid pressure applied by the secondary fluid is removed and the anti-backflow device 238 automatically moves back to the closed position (of FIG. 2A) due to its compliant nature and the aforementioned configuration, thereby preventing any appreciable amount of primary fluid from passing through the anti-backflow device 238 into the fluid injection device 216. Thus, because the primary fluid is sealed off from entering the access port opening 234 in this manner, the fluid injection device 216 can remain connected to the access port body 226 without the concern of diluting the secondary fluid with the primary fluid. Then, after a time period passes when the patient requires another dosage of the secondary fluid, the clinician can again apply pressure to the plunger of the fluid injection device 216 to inject a second dosage of the secondary fluid through the anti-backflow device 238 and into the primary fluid path P2 to mix with the primary fluid for delivery to the patient. This process can be repeated for successive dosages using the same fluid injection device 216 while being continuously connected to the access port body 226. In addition, when another fluid injection device is needed for delivery of another secondary fluid to the patient, the fluid injection device 216 can be removed from the unidirectional access port assembly 218, and then the access port body 226 can be sterilized or disinfected (e.g., swabbed or cleaned with a swab tool) to be ready to receive a subsequent fluid injection device containing a secondary fluid. Thus, the access port body 226 can be sterilized or otherwise cleaned (e.g., can be swab-able (able to be swabbed)), but also permits continuous connection of a particular fluid injection device over multiple, successive dosages of a secondary fluid to the patient.

FIG. 3A illustrates an example unidirectional access port assembly 318 in a closed position and coupled to a fluid injection device 316, and FIG. 3B illustrates the unidirectional access port assembly 318 in an open position. The unidirectional access port assembly 318 can be structurally and functionally similar to that of the unidirectional access port assembly 218, which should be readily appreciated from FIGS. 2A-3B and the associated descriptions.

In this example, the unidirectional access port assembly 318 comprises a primary flow body 320 comprising a sidewall 322 and opposing first and second openings 324a and 324b. A flow channel 325 extends between the first and second openings 324a and 324b. The primary flow body 320 can be coupled to respective primary flow line segments (e.g., 18e and 18f of FIGS. 1A and 1B, or 108c and 108d of FIG. 1C) via respective first and second openings 324a and 324b, such as by a medical grade adhesive. Thus, the unidirectional access port assembly 318 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The unidirectional access port assembly 318 can comprise an access port body 326 supported at least partially by the primary flow body 320. The access port body 326 extends outwardly and orthogonally from the sidewall 322 to form a generally T-shape configuration. In another example, the access port body 326 can comprise a Y-shape configuration. The access port body 326 can be generally cylindrically shaped having one end attached (e.g., glued, ultrasonically welded, or press fit) into a side opening 328 of the primary flow body 320. On the other end, the access port body 326 comprises a male type connection portion 330 configured to removably couple a female type connector portion 332 of a fluid injection device 316, such as a male-female Luer lock configuration. The access port body 326 further comprises an access port opening 334 that defines an access port 314 and that receives an injection end 335 of the fluid injection device 316 for delivery of a secondary fluid from a fluid chamber (not shown) of the fluid injection device 316 into the access port 314 via a secondary fluid path S3 of the fluid injection device 316.

The unidirectional access port assembly 318 can further comprise an anti-backflow device 338 supported at least partially by the access port body 326, and that facilitates fluid communication to a primary fluid path P3 (of an IV set) via the access port 314. More specifically, the anti-backflow device 338 can comprise an elastomeric component that fluidly separates the access port 314 from the flow channel 325, and which can comprise a one-way compliant opening 336 operable to prevent the primary fluid from passing through the one-way compliant opening 336 into the fluid chamber of the fluid injection device 316 while the fluid injection device 316 is removably coupled to the access port body 326. In this example, the anti-backflow device 338 can be similar to a “duck-bill” elastomeric check valve, such that the compliant opening 336 is a slit formed through the anti-backflow device 338. Thus, in the closed position of FIG. 3A, the one-way compliant opening 336 prevents or restricts the primary fluid from passing into the access port opening 334, because the elastomeric properties of the anti-backflow device 338 are such that the anti-backflow device 338 is rigid enough to overcome the fluid pressure applied by the primary fluid in the primary fluid path P3, and because the compliant opening 336 is formed as a slit so that the primary fluid cannot pass through without sufficient fluid pressure. In the open position of FIG. 3B, the one-way compliant opening 336 permits or allows the secondary fluid to pass from the fluid injection device 316 through the access port opening 334 and into the primary fluid path P3, because the elastomeric properties of the anti-backflow device 338 are such that the anti-backflow device 338 is compliant enough to give way to the fluid pressure of the secondary fluid applied by the fluid injection device 316 on one side of the anti-backflow device 338 such that the slit opens up and protrudes inwardly towards the primary fluid path for mixing the secondary fluid with the primary fluid.

In the example shown in FIGS. 3A and 3B, the primary flow body 320 and the access port body 326 cooperate to retain and support a perimeter portion 340 of the anti-backflow device 338. More specifically, the primary flow body 320 can comprise a shoulder portion 342 that supports a corner portion of the perimeter portion 340 of the anti-backflow device 338, and the access port body 326 can comprise a complimentary shoulder portion 344 that supports an opposing corner portion of the perimeter portion 340. Thus, the perimeter portion 340 of the anti-backflow device 338 can be somewhat sandwiched or press fit between respective shoulder portions 342 and 344 to axially and radially support the anti-backflow device 338. A medical grade adhesive may be applied between the perimeter portion 340 and the shoulder portions 342 and 344 to fix or attach the anti-backflow device 338 in-place. Thus, the anti-backflow device 338 fluidly separates the access port 314 from the flow channel 325 when in the closed position, as in FIG. 3A.

In this example, the anti-backflow device 338 can have a beveled shape or configuration. More specifically, the perimeter portion 340 can be an annular ring portion extending parallel relative to a longitudinal central axis X3 of the primary flow body 320. A beveled portion 341 can transition inwardly from the perimeter portion 340 and toward the primary fluid path P3 (i.e., away from the injection end 335), such that the beveled portion 341 is formed at an angle relative to the longitudinal central axis X3 of the primary flow body 320, so that the beveled portion 341 generally forms a conical space proximate the injection end 335 of the fluid injection device 316.

Notably, the injection end 335 of the fluid injection device 316 can be positioned very near one side of the anti-backflow device 338, such as 1 to 2 millimeters to account for tolerances between the fluid injection device 316 and the access port body 326. Thus, any possible “dead-space” is minimized because the injection end 335 can be near the anti-backflow device 338. Ideally, the injection end 335 can slightly touch the surface of the beveled portion 341 of the anti-backflow device 338 when in the closed position (as illustrated in FIG. 3A), thereby eliminating dead-space on this side of the anti-backflow device 338. On the other side of the anti-backflow device 338 adjacent the primary fluid path P3, the anti-backflow device 338 can comprise a similarly shaped beveled portion 343 that extends outwardly at an angle and into the primary fluid path P3. Thus, the beveled portion 343 is positioned proximate or within the primary fluid path P3 to eliminate dead-space on this side of the anti-backflow device 338. More particularly, the sidewall 322 can comprise an upper planar sidewall portion 346 and a lower planar sidewall portion 348 that define a flow plane 350 along which the primary fluid generally flows through the flow channel 325. When in the open position, the one-way compliant opening 336 extends beyond this flow plane 350 toward the longitudinal central axis X3, so that the secondary fluid can be injected and mixed with the primary fluid at a mixing region that is within the primary fluid path P3.

As detailed above regarding the unidirectional access port assembly 218 shown in FIGS. 2A and 2B, the unidirectional access port assembly 318 can be similarly operable to maintain continuous connection to the fluid injection device 316 over multiple successive dosages of the secondary fluid from the fluid injection device 316 into the primary fluid path P3 through the anti-backflow device 338. This is because, when pressure is removed from a plunger (or other movable device) of the fluid injection device 316 (after a first dosage is injected, as in FIG. 3B), fluid pressure applied by the secondary fluid is removed and the anti-backflow device 338 automatically moves back to the closed position (of FIG. 3A) due to its compliant nature and the aforementioned configuration, thereby preventing any appreciable amount of primary fluid from passing through the anti-backflow device 338 into the fluid injection device 316. Thus, because the primary fluid is sealed off from entering the access port opening 334 in this manner, the fluid injection device 316 can remain connected to the access port body 326 without the concern of diluting the secondary fluid with the primary fluid. Then, after a time period passes when the patient requires another dosage of the secondary fluid, the clinician can again apply pressure to a plunger of the fluid injection device 316 to inject a second dosage of the secondary fluid through the anti-backflow device 338 and into the primary fluid path P3 to mix with the primary fluid for delivery to the patient. This process can be repeated for successive dosages using the same fluid injection device 316 while being continuously connected to the access port body 326. In addition, when another fluid injection device is needed for delivery of another secondary fluid to the patient, the fluid injection device 316 can be removed from the unidirectional access port assembly 318, and then the access port body 326 can be swabbed or cleaned with a swab tool to be ready to receive a new fluid injection device. Thus, the access port body 326 is swab-able, but also permits continuous connection of a particular fluid injection device over multiple, successive dosages of a secondary fluid to the patient.

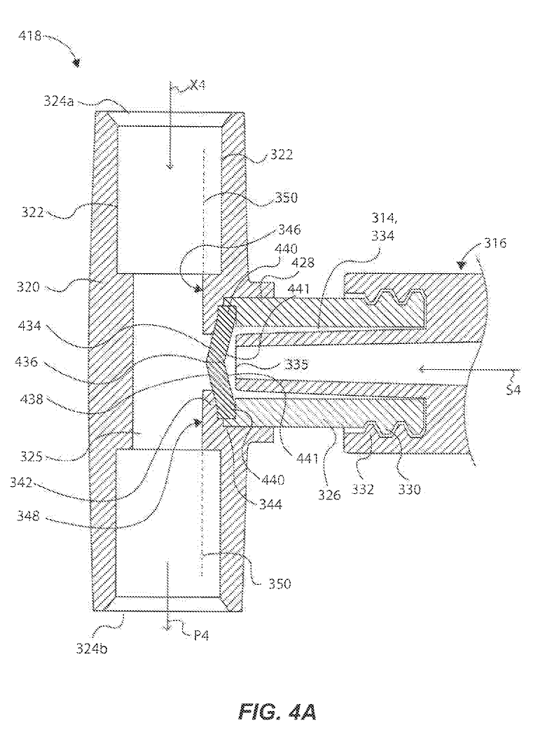

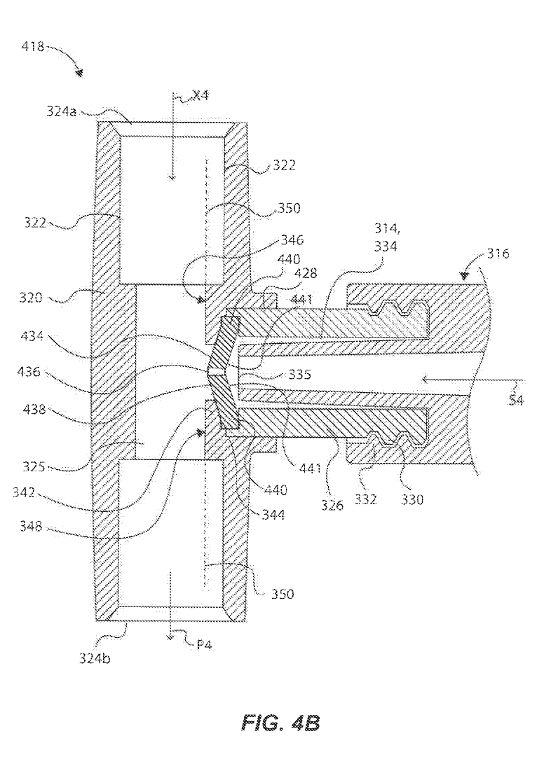

FIG. 4A illustrates an example unidirectional access port assembly 418 in a closed position and coupled to a fluid injection device 416, and FIG. 4B shows the unidirectional access port assembly 418 in an open position. The unidirectional access port assembly 418 can be structurally and functionally similar to that of the unidirectional access port assembly 318 of FIG. 3A, so it will not be discussed in as much detail, but such similarities should readily be appreciated from the examples of FIGS. 2A-3B and the associated descriptions.

In this example, the unidirectional access port assembly 418 can have the same primary flow body 320 and the access port body 326 of FIG. 3A, so the reference labels have been duplicated on FIGS. 4A and 4B as being the same components as in FIG. 3A. Here, an anti-backflow device 438 is supported at least partially by the access port body 326, and facilitates fluid communication to a primary fluid path P4 (of an IV set) via the access port 314. More specifically, the anti-backflow device 438 can comprise an elastomeric component that fluidly separates the access port 314 from the flow channel 325, and which can comprise a one-way compliant opening 436 operable to prevent the primary fluid from passing through the one-way compliant opening 436 into a fluid chamber of the fluid injection device 316 while the fluid injection device 316 is removably coupled to the access port body 326. In this example, the anti-backflow device 438 can be similar to a “duck-bill” elastomeric check valve, such that the compliant opening 436 is a slit formed through the anti-backflow device 438. Thus, in the closed position of FIG. 4A, the one-way compliant opening 436 prevents or restricts the primary fluid from passing into the access port opening 334, because the elastomeric properties of the anti-backflow device 438 are such that the anti-backflow device 438 is rigid enough to overcome the fluid pressure applied by the primary fluid in the primary fluid path P2, and because the compliant opening 436 is formed as a slit. This is also because of the outward tapered shape of the anti-backflow device 438 that comes to a point or tip area at or near the compliant opening 436. Notably, this outward tapered shape extends away from the injection end 335, and towards the longitudinal central axis X4 of the primary flow body 320. In the open position of FIG. 4B, the one-way compliant opening 436 permits or allows the secondary fluid to pass from the fluid injection device 416 through the access port opening 334 and into the primary fluid path P4, because the elastomeric properties of the anti-backflow device 438 are such that the anti-backflow device 438 is compliant enough to give way to the fluid pressure of the secondary fluid applied by the fluid injection device 416 on one side of the anti-backflow device 438 such that the slit or compliant opening 436 is pushed outwardly and opens up, and therefore protrudes inwardly towards the primary fluid path for mixing the secondary fluid with the primary fluid, in a similar manner as described regarding FIG. 3B.

The primary flow body 320 and the access port body 326 cooperate to retain and support a circumferential perimeter portion 440 of the anti-backflow device 438. Thus, the circumferential perimeter portion 440 of the anti-backflow device 438 can be somewhat sandwiched or press fit or supported between respective shoulder portions 342 and 344 to axially and radially support the anti-backflow device 438, so that the circumferential perimeter portion 440 remains relatively static between the open and closed positions, while a central area (including the compliant opening 436) can deflect or deform upon the application or removal of fluid pressure via the injection end 335. A medical grade adhesive may be applied between the perimeter portion 440 and the shoulder portions 342 and 344 to fix or attach the anti-backflow device 338 in-place. Thus, the anti-backflow device 438 fluidly separates the access port 314 from the flow channel 325 when in the closed position, as shown in FIG. 4A.

In this example, the anti-backflow device 438 can have a beveled shape configuration. More specifically, a beveled portion 441 can transition inwardly from the perimeter portion 440 and toward the primary fluid path P4 (i.e., away from the injection end 335), such that the beveled portion 441 is formed at an angle relative to the longitudinal central axis X4 of the primary flow body 320, so that the beveled portion 441 generally forms a conical space proximate the injection end 335 of the fluid injection device 316.

Notably, the injection end 335 of the fluid injection device 316 can be positioned very near one side of the anti-backflow device 438, such as 1 to 2 millimeters to account for tolerances between the fluid injection device 316 and the access port body 326. Thus, any possible “dead-space” is minimized. Ideally, the injection end 335 can slightly touch the surface of the beveled portion 441 of the anti-backflow device 438 when in the closed position (as illustrated in FIGS. 4A and 4B), thereby eliminating dead-space on this side of the anti-backflow device 438. On the other side of the anti-backflow device 438 adjacent the primary fluid path P4, the anti-backflow device 438 can comprise a similarly shaped beveled portion 443 that extends outwardly at an angle and into the primary fluid path P4. Thus, the beveled portion 443 is positioned proximate or within the primary fluid path P4 to eliminate dead-space on this side of the anti-backflow device 438. Thus, the one-way compliant opening 436 can extends toward the flow plane 350 toward the longitudinal central axis X4, so that the secondary fluid can be injected and mixed with the primary fluid at a mixing region that is within the primary fluid path P4.

As detailed above regarding the examples of FIGS. 2-3B, the unidirectional access port assembly 418 is similarly operable to maintain continuous connection to the fluid injection device 316 over multiple successive dosages of the secondary fluid from the fluid injection device 316 into the primary fluid path P4 through the anti-backflow device 438.

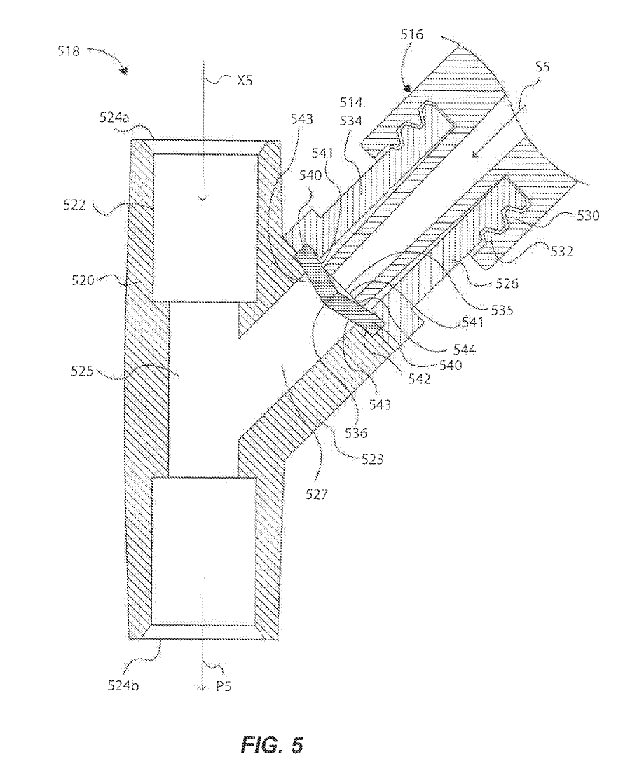

FIG. 5 illustrates an example unidirectional access port assembly 518 in a closed position and coupled to a fluid injection device 516. The unidirectional access port assembly 518 can be similar to that of the unidirectional access port assembly 418 of FIG. 4A, but FIG. 5 illustrates a Y-site type connector configuration.

In this example, the unidirectional access port assembly 518 comprises a primary flow body 520 comprising a sidewall 522 and opposing first and second openings 524a and 524b. A flow channel 525 extends between the first and second openings 524a and 524b. The primary flow body 520 can be coupled to respective primary flow line segments (e.g., 18e and 18f of FIGS. 1A and 1B, and 108b and 108c of FIG. 1C) via respective first and second openings 524a and 524b, such as by a medical grade adhesive. Thus, the unidirectional access port assembly 518 at least partially defines the primary flow line and the primary fluid path of an IV set, such as the IV set 13 of FIG. 1B, and the IV set 106 of FIG. 1C.

The unidirectional access port assembly 518 can comprise an access port body 526 supported at least partially by the primary flow body 520. The access port body 526 extends outwardly and transverse from a sidewall portion 523 to form generally a Y-shape configuration (a T-shape configuration also being contemplated). The sidewall portion 523 can define a supplemental fluid channel 527 in fluid communication with the fluid channel 525. The access port body 526 can be generally cylindrically shaped having one end attached (e.g., glued) to an end of the sidewall portion 523 of the primary flow body 520. On the other end, the access port body 526 comprises a male type connection portion 530 configured to removably couple a female type connector portion 532 of a fluid injection device 516, such as a male-female luer lock configuration. The access port body 526 further comprises an access port opening 534 that defines an access port 514 and that receives an injection end 535 of the fluid injection device 516 for delivery of a secondary fluid from a fluid chamber (not shown) of the fluid injection device 516 into the access port 514 via a secondary fluid path S5 of the fluid injection device 516.

The unidirectional access port assembly 518 can further comprise an anti-backflow device 538 supported at least partially by the access port body 526, and that facilitates fluid communication to a primary fluid path P5 (of an IV set) via the access port 514. More specifically, the anti-backflow device 538 can comprise an elastomeric component that fluidly separates the access port 514 from the supplemental flow channel 527 (and flow channel 525) The anti-backflow device 538 can comprise a one-way compliant opening 536 operable to prevent the primary fluid from passing through the one-way compliant opening 536 into the fluid chamber of the fluid injection device 516 while the fluid injection device 516 is removably coupled to the access port body 526. In this example, the anti-backflow device 538 can be similar to a “duck-bill” elastomeric check valve, such that the compliant opening 536 is a slit formed through the anti-backflow device 538, similar to the configuration of the anti-backflow device 438 of FIG. 4A. Thus, in the closed position of FIG. 5, the one-way compliant opening 536 prevents or restricts the primary fluid from passing into the access port opening 534, because the elastomeric properties of the anti-backflow device 538 are such that the anti-backflow device 538 is rigid enough to overcome the fluid pressure applied by the primary fluid in the primary fluid path P5, and because the compliant opening 536 is formed as a slit. In the open position (not shown), the one-way compliant opening 536 permits or allows the secondary fluid to pass from the fluid injection device 516 through the access port opening 534 and into the primary fluid path P5, because the elastomeric properties of the anti-backflow device 538 are such that the anti-backflow device 538 is compliant enough to give way to the fluid pressure of the secondary fluid applied by the fluid injection device 516 on one side of the anti-backflow device 538 such that the slit opens up and protrudes inwardly towards the primary fluid path for mixing the secondary fluid with the primary fluid.

The primary flow body 520 and the access port body 526 cooperate to retain and support a perimeter portion 540 of the anti-backflow device 538. More specifically, the primary flow body 520 can comprise a shoulder portion 542 that supports a corner portion of the perimeter portion 540 of the anti-backflow device 538, and the access port body 526 can comprise a complimentary shoulder portion 544 that supports an opposing corner portion of the perimeter portion 540. Thus, the perimeter portion 540 of the anti-backflow device 538 can be somewhat sandwiched or press fit between respective shoulder portions 542 and 544 to axially and radially support the anti-backflow device 538. A medical grade adhesive may be applied between the perimeter portion 540 and the shoulder portions 542 and 544 to fix or attach the anti-backflow device 538 in-place. Thus, the anti-backflow device 538 fluidly separates the access port 514 from the flow channel 525 when in the closed position, as in FIG. 5.

In this example, the anti-backflow device 538 can have a beveled shape or configuration. More specifically, a beveled portion 541 can transition inwardly from the perimeter portion 540 and toward the primary fluid path P5 (i.e., away from the injection end 535), such that the beveled portion 541 is formed at an angle relative to the longitudinal central axis X5 of the primary flow body 520, so that the beveled portion 541 generally forms a conical space proximate the injection end 535 of the fluid injection device 516. A central axis extending through the one-way compliant opening 536 can be transverse to the longitudinal axis X5 of the primary flow body 520, due to the Y-shaped configuration of the unidirectional access port assembly 518. Thus, the secondary fluid can be introduced into the supplemental fluid channel 527 and into the fluid channel 525 at a downward angle such that gravity can assist to downwardly carry the secondary fluid into the primary fluid, which helps to prevent secondary fluid from gathering or staying within the supplemental fluid channel 527 and not reaching the patient.

Notably, the injection end 535 of the fluid injection device 516 can be positioned very near one side of the anti-backflow device 538, such as 1 to 2 millimeters to account for tolerances between the fluid injection device 516 and the access port body 526. Thus, any possible “dead-space” is minimized because the injection end 235 can be near the anti-backflow device 238. Ideally, the injection end 535 can slightly touch the surface of the beveled portion 541 of the anti-backflow device 538 when in the closed position (as illustrated in FIG. 5), thereby eliminating dead-space on this side of the anti-backflow device 538.

As detailed above regarding the examples of FIGS. 2-4B, the unidirectional access port assembly 518 is similarly operable to maintain continuous connection to the fluid injection device 516 over multiple successive dosages of the secondary fluid from the fluid injection device 516 into the primary fluid path P5 through the anti-backflow device 538.

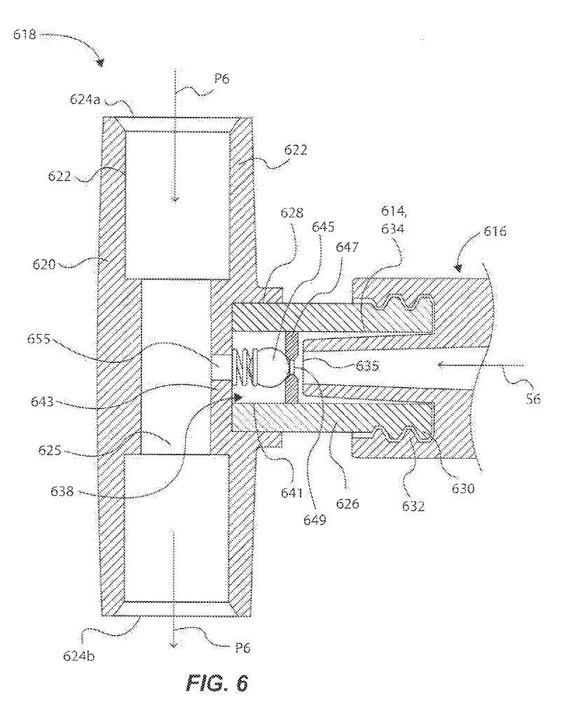

FIG. 6 illustrates an example unidirectional access port assembly 618 in a closed position and coupled to a fluid injection device 616. The unidirectional access port assembly 618 comprises a primary flow body 620 comprising a sidewall 622 and opposing first and second openings 624a and 624b. A flow channel 625 extends between the first and second openings 624a and 624b. The primary flow body 620 can be coupled to respective primary flow line segments (e.g., 18e and 18f of FIGS. 1A and 1B, or 108c and 108d of FIG. 1C) via respective first and second openings 624a and 624b, such as by an adhesive. Thus, the unidirectional access port assembly 618 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The unidirectional access port assembly 618 can comprise an access port body 626 supported at least partially by the primary flow body 620. The access port body 626 extends outwardly and orthogonally from the sidewall 622 to form generally a T-shape configuration. In another example, the access port body 626 can comprise a Y-shape configuration. The access port body 626 can be generally cylindrically shaped having one end attached (e.g., glued, ultrasonically welded, or press fit) into a side opening 628 of the primary flow body 620. On the other end, the access port body 626 comprises a male type connection portion 630 configured to removably couple a female type connector portion 632 of a fluid injection device 616, such as a male-female luer lock configuration. The access port body 626 further comprises an access port opening 634 that defines an access port 614 and that receives an injection end 635 of the fluid injection device 616 for delivery of a secondary fluid from a fluid chamber (not shown) of the fluid injection device 616 into the access port 614 via a secondary fluid path S6 of the fluid injection device 616.

The unidirectional access port assembly 618 can further comprise an anti-backflow device 638 supported at least partially by the access port body 626, and that facilitates fluid communication to a primary fluid path P6 (of an IV set) via the access port 614. More specifically, the anti-backflow device 638 can comprise a ball type check valve that fluidly separates the access port 614 from the flow channel 625, and that is operable to prevent the primary fluid from passing through the anti-backflow device 638 into a fluid chamber of the fluid injection device 616 while the fluid injection device 616 is removably coupled to the access port body 626.

In one example, the anti-backflow device 638 can comprise a spring 641 (e.g., a coil spring) seated at one end against a spring support portion 643 of the primary flow body 620. The spring support portion 643 can define a secondary flow aperture 655 through which the secondary fluid can flow from the fluid injection device 616 when the anti-backflow device 638 is opened by fluid pressure from the secondary fluid. The spring 641 may be seated by other means, such as in notches or protrusions of the spring support portion 643 adjacent the end of the spring 641. On the other end, the spring 641 supports and seats a ball 645. Specifically, in the closed position in FIG. 6, the spring 641 biases the ball 645 toward a ball support portion 647, which can comprise a seat opening 649 through which the secondary fluid passes when the anti-backflow device 638 is opened by fluid pressure exerted by secondary fluid injected by the fluid injection device 616. Thus, in the closed position, the anti-backflow device 638 prevents or restricts the primary fluid from passing into the access port opening 634, because the spring 641 is stiff enough to appropriately seat the ball 645 against the ball support portion 647 to close off the seat opening 649 to prevent backflow of the primary fluid into the secondary fluid. In the open position (not shown), the anti-backflow device 638 permits or allows the secondary fluid to pass from the fluid injection device 616 through the seat opening 649 and into the primary fluid path P6, because the spring 641 is compliant enough to give way to the fluid pressure of the secondary fluid applied by the fluid injection device 616. Thus, fluid pressure from the secondary fluid applies pressure to one side of the ball 645 through the seat opening 649, thereby causing compression of the spring 641 while unseating the ball 645 from the ball support portion 647, thereby exposing the seat opening 649 so that the secondary fluid can flow from the fluid injection device 616 through the seat opening 649 and around the ball 645 and through the secondary flow aperture 655 and into the primary fluid path P6. As illustrated, the components of the anti-backflow device 638 are entirely enclosed and supported within the access port opening 634 of the access port body 626, which helps to ensure sterility of the moving parts of the ball check valve.

Notably, the injection end 635 of the fluid injection device 616 can be positioned very near the ball support portion 647, such as 1 to 2 millimeters (as illustrated in FIG. 6), to account for tolerances between the coupling of the fluid injection device 616 to the access port body 626. Thus, any possible “dead-space” is minimized. Ideally, the injection end 635 can slightly touch the side surface of the ball support portion 647, thereby eliminating dead-space on this side of the anti-backflow device 638.

Similar to the other unidirectional access port assemblies discussed herein, the unidirectional access port assembly 618 is operable to maintain continuous connection to the fluid injection device 616 over multiple successive dosages of the secondary fluid from the fluid injection device 616 into the primary fluid path P6 through the anti-backflow device 638. This is because, when pressure is removed from a plunger (or other movable device) of the fluid injection device 616 (after a first dosage is injected), fluid pressure from applied by secondary fluid is removed from the system, and the anti-backflow device 638 automatically moves back to the closed position (FIG. 6) when the spring 641 releases its potential energy to appropriately seat the ball 645 against the ball support portion 647, thereby preventing any primary fluid from passing through the seat opening 649 into the fluid injection device 616. Thus, the fluid injection device 616 can remain connected to the access port body 626 without the concern of diluting the secondary fluid with the primary fluid. Then, after a time period passes when the patient requires another dosage of the secondary fluid, the clinician can again apply pressure to the plunger of the fluid injection device 616 to inject a second dosage of the secondary fluid through the anti-backflow device 638 and into the primary fluid path P6 to mix with the primary fluid for delivery to the patient. This process can be repeated for successive dosages using the same fluid injection device 616 while being continuously connected to the access port body 626. In addition, when another fluid injection device is needed for delivery of another secondary fluid to the patient, the fluid injection device 616 can be removed from the unidirectional access port assembly 618, and then the access port body 626 can be swabbed or cleaned with a swab tool to be ready to receive a new fluid injection device. Thus, the access port body 626 is swab-able, but also permits continuous connection of a fluid injection device over multiple, successive dosages of a secondary fluid into a primary fluid.

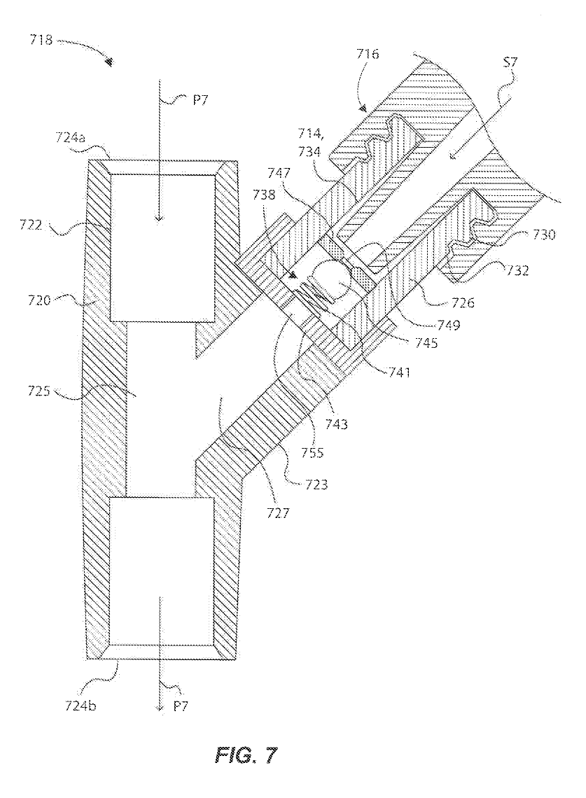

FIG. 7 illustrates an example unidirectional access port assembly 718 in a closed position and coupled to a fluid injection device 716. The unidirectional access port assembly 718 is similar in functionality to the unidirectional access port assembly 618 of FIG. 6, but here, the unidirectional access port assembly 718 is in a Y-shaped configuration, such as similar to Y-site IV ports. Specifically, the unidirectional access port assembly 718 comprises a primary flow body 720 comprising a sidewall 722 and opposing first and second openings 724a and 724b. A flow channel 725 extends between the first and second openings 724a and 724b. The primary flow body 720 can be coupled to respective primary flow line segments (e.g., 18e and 18f of FIGS. 1A and 1B, or 108b and 108c of FIG. 1C) via respective first and second openings 724a and 724b, such as by an adhesive. Thus, the unidirectional access port assembly 718 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The unidirectional access port assembly 718 can comprise a first access port body 726a and a second access port body 726b coupled to each other to form a secondary access port body coupled to a primary access port body 723 of the primary flow housing 720. The second access port body 726b extends outwardly and transverse from the primary access port body 723 to generally form a Y-shape configuration. The primary access port body 723 can define a supplemental fluid channel 727 in fluid communication with the fluid channel 725. The second access port body 726b can be generally cylindrically shaped having one end attached (e.g., glued) to an inner opening of the first access port body 726a, which is attached to an end of the primary access port body 723 of the primary flow body 720, as illustrated. On the other end, the second access port body 726b comprises a male type connection portion 730 configured to removably couple a female type connector portion 732 of a fluid injection device 716. The second access port body 726b further comprises an access port opening 734 that defines an access port 714 and that receives an injection end 735 of the fluid injection device 716 for delivery of a secondary fluid from a fluid chamber (not shown) of the fluid injection device 716 into the access port 714 via a secondary fluid path S7 of the fluid injection device 716.

The unidirectional access port assembly 718 can further comprise an anti-backflow device 738 supported at least partially by the second access port body 726b, and that facilitates fluid communication to a primary fluid path P7 (of an IV set) via the access port 714. More specifically, the anti-backflow device 738 can comprise a ball type check valve that fluidly separates the access port 714 from the supplemental flow channel 727 (and flow channel 725). The anti-backflow device 738 is operable to prevent the primary fluid from passing through the anti-backflow device 738 into the fluid chamber of the fluid injection device 716 while the fluid injection device 716 is removably coupled to the second access port body 726b.

In this example, the anti-backflow device 738 can comprise a spring 741 (e.g., a coil spring) seated at one end against a spring support portion 743 of the primary flow body 720. The spring support portion 743 can define a secondary flow aperture 755 through which the secondary fluid can flow from the fluid injection device 716 when the anti-backflow device 738 is opened by fluid pressure via the secondary fluid. The spring 741 may be seated by other means, such as in notches or protrusions of the spring support portion 743 adjacent the end of the spring 741. On the other end, the spring 741 supports and seats a ball 745. Specifically, in the closed position of FIG. 7, the spring 741 biases the ball 745 toward a ball support portion 747, which can comprise a seat opening 749 through which the secondary fluid passes when the anti-backflow device 738 is opened by fluid pressure exerted secondary fluid injected by the fluid injection device 716. Thus, in the closed position, the anti-backflow device 738 prevents or restricts the primary fluid from passing into the access port opening 734, because the spring 741 is stiff enough to appropriately seat the ball 745 against the ball support portion 747 to close off the seat opening 749 to prevent backflow of the primary fluid into the secondary fluid. In the open position (not shown), the anti-backflow device 738 permits or allows the secondary fluid to pass from the fluid injection device 716 through the seat opening 749 and into the primary fluid path P7, because the spring 741 is compliant enough to give way to the fluid pressure of the secondary fluid applied by the fluid injection device 716. Thus, fluid pressure from the secondary fluid applies pressure to one side of the ball 745 through the seat opening 749, thereby causing compression of the spring 741 while unseating the ball 745 from the ball support portion 747, thereby exposing the seat opening 749 so that the secondary fluid can flow through the seat opening 749 and around the ball 745 and through the secondary flow aperture 755 and into the primary fluid path P7. As illustrated, the components of the anti-backflow device 738 are entirely enclosed and supported within the access port opening 734 of the second access port body 726b, which helps to ensure sterility of the moving parts of the ball check valve.

Notably, the injection end 735 of the fluid injection device 716 can be positioned very near the ball support portion 747, such as 1 to 2 millimeters (as illustrated in FIG. 7), to account for tolerances between the coupling of the fluid injection device 716 to the second access port body 726b. Thus, any possible “dead-space” is minimized. Ideally, the injection end 735 can slightly touch the side surface of the ball support portion 747, thereby eliminating dead-space on this side of the anti-backflow device 738.

A central axis extending through the ball 745 and the secondary flow aperture 755 can be transverse to a longitudinal axis of the primary flow body 720, due to the Y-shaped configuration of the unidirectional access port assembly 718. Thus, the secondary fluid can be introduced into the supplemental fluid channel 727 and into the fluid channel 725 at a downward angle such that gravity can assist to carry the secondary fluid into the primary fluid downwardly, which helps to prevent secondary fluid from gathering or staving within the supplemental fluid channel 727 and not reaching the patient.

Similarly as discussed regarding FIG. 6, the unidirectional access port assembly 718 is operable to maintain continuous connection to the fluid injection device 716 over multiple successive dosages of the secondary fluid from the fluid injection device 716 into the primary fluid path P7 through the anti-backflow device 738.

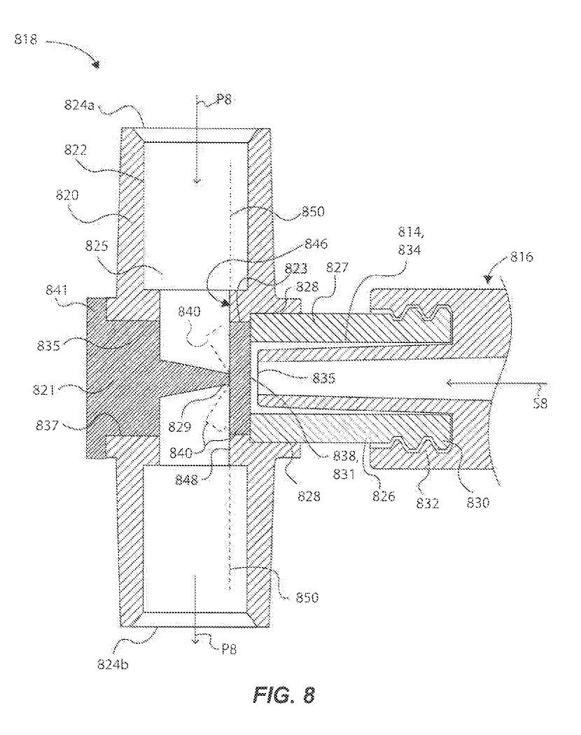

FIG. 8 illustrates an example unidirectional access port assembly 818 in a closed position and coupled to a fluid injection device 816 (with shadow lines illustrating an open position). The unidirectional access port assembly 818 comprises a primary flow body 820 comprising a sidewall 822 and opposing first and second openings 824a and 824b. A flow channel 825 extends between the first and second openings 824a and 824b. The primary flow body 820 can be coupled to respective primary flow line segments (e.g., 18e and 18f of FIGS. 1A and 1B, or 108c and 108d of FIG. 1C) via respective first and second openings 824a and 824b, such as by an adhesive. Thus, the unidirectional access port assembly 818 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The unidirectional access port assembly 818 can comprise an access port body 826 supported at least partially by the primary flow body 820. The access port body 826 extends outwardly and orthogonally from the sidewall 822 to form generally a T-shape configuration. In another example, the access port body 826 can comprise a Y-shape configuration. The access port body 826 can be generally cylindrically shaped having one end attached (e.g., glued) to a side opening 828 of the primary flow body 820. On the other end, the access port body 826 comprises a male type connection portion 830 configured to removably couple a female type connector portion 832 of a fluid injection device 816. The access port body 826 further comprises an access port opening 834 that defines an access port 814 and that receives an injection end 835 of the fluid injection device 816 for delivery of a secondary fluid from a fluid chamber (not shown) of the fluid injection device 816 into the access port 814 via a secondary fluid path S8 of the fluid injection device 816.

The unidirectional access port assembly 818 can further comprise an anti-backflow device 838 supported by the access port body 826 and the primary flow body 820, and that facilitates fluid communication to a primary fluid path P8 (of an IV set) via the access port 814. More specifically, the anti-backflow device 838 can comprise an elastomeric component that fluidly separates the access port 814 from the flow channel 825. As detailed below, the anti-backflow device 838 is operable to prevent the primary fluid from passing from the channel 825 and into a fluid chamber of the fluid injection device 816 while the fluid injection device 816 is removably coupled to the access port body 826.

The anti-backflow device 838 can be supported by the access port body 826, the primary flow body 820, and a valve support device 821. More specifically, a perimeter portion 840 of the anti-backflow device 838 can be seated in a valve opening 823 of the primary flow body 820 to radially support the anti-backflow device 838. On one side of the anti-backflow device 838, the perimeter portion 840 is axially supported by an end portion 827 of the access port body 826. On the other side of the anti-backflow device 838, a support end 829 of the valve support device 821 interfaces with and supports a central area 831 of the anti-backflow device 838. The valve support device 821 can have cylindrical insert portion 835 that is attached to a side aperture 837 of the primary flow body 820. An attachment flange 841 can be coupled to an outer surface of the primary flow body 820 to maintain the position of the valve support device 821. The support end 829 can be frustoconically shaped (or it can comprise other shapes) and can extend through the flow channel 825 and interface with, or bias, the anti-backflow device 838 into its seated position against the end 827 of the access port body 826. As shown in the closed position of FIG. 8, the anti-backflow device 838 can be a planar disk that is cylindrically shaped.

In this example, the central area 831 is fixed in-place by the support end 829, in a manner such that the perimeter portion 840 is movable or compliant to allow passage of fluid when fluid pressure is applied via the secondary fluid from the fluid injection device 816. Accordingly, upon applying fluid pressure via the fluid injection device 816, the secondary fluid is dispensed out the injection end 835 and applies pressure to one side of the anti-backflow device 838, so that the perimeter portion 840 flexes inwardly toward the primary fluid path P8, as shown by the shadow lines of the perimeter portion 840, illustrating the open position. In this position, the secondary fluid is permitted to pass about and over and through the anti-backflow device 838. Once fluid pressure is removed via the fluid injection device 816, the perimeter portion 840 automatically moves back to the closed position of FIG. 8, due to the compliant nature of the anti-backflow device 838.

Notably, the injection end 835 of the fluid injection device 816 can be positioned very near one side of the anti-backflow device 838, such as 1 to 2 millimeters (as illustrated in FIG. 8), to account for tolerances between the coupling of the fluid injection device 816 to the access port body 826. Thus, any possible “dead-space” is minimized. On the other side of the anti-backflow device 838 adjacent the primary fluid path P8, the anti-backflow device 838 is positioned proximate or near the primary fluid path P8 to reduce or minimize dead-space on this side of the anti-backflow device 838. More specifically, the sidewall 822 can comprise an upper planar sidewall portion 846 and a lower planar sidewall portion 848 that define a flow plane 850 along which the primary fluid generally flows through the flow channel 825. One planar side of the anti-backflow device 838 is collinear with this flow plane 850, or is flush with the first and second sidewall portions 846. When in the open position, the perimeter portion 840 of the anti-backflow device 838 extends inwardly beyond this flow plane 850, so that the secondary fluid can be injected and mixed with the primary fluid at a mixing region that is generally within the primary fluid path P8. Thus, proximate this side of the anti-backflow device 838, any possible dead-space is eliminated to ensure proper delivery of the secondary fluid to the patient.

Similarly as exemplified above in other examples, the unidirectional access port assembly 818 is operable to maintain continuous connection to the fluid injection device 816 over multiple successive dosages of the secondary fluid from the fluid injection device 816 into the primary fluid path P8 through the anti-backflow device 838.

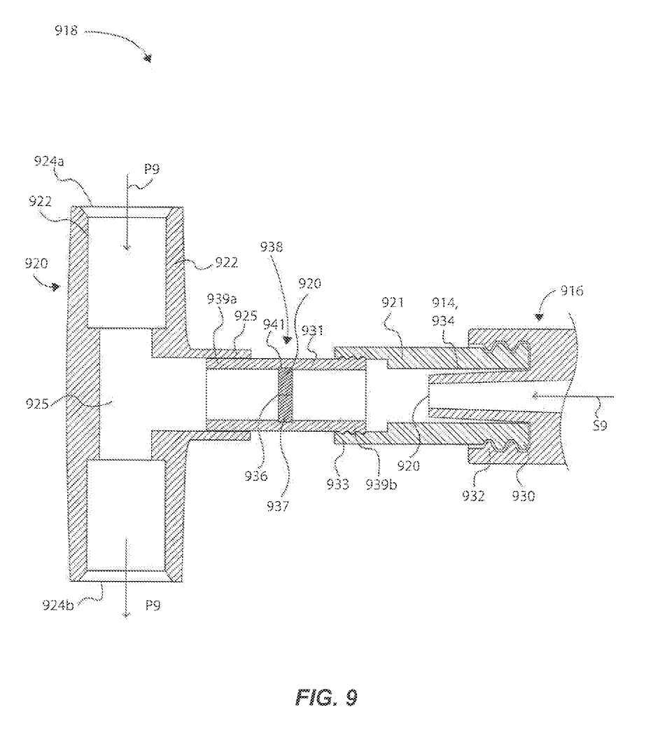

FIG. 9 illustrates an example unidirectional access port assembly 918 in a closed position and coupled to a fluid injection device 916. The unidirectional access port assembly 918 can comprise a primary flow body 920, an anti-backflow device 938, and a connector device 921. These components can be coupled to each other in the manner shown, such that the anti-backflow device 938 is generally situated between the primary flow body 920 and the connector device 921. The fluid injection device 916 can be removably coupled to the connector device 921, such as via a male type connection portion 930 interfaced with a female type connector portion 932 of the fluid injection device 916. In one example, the connector device 921 can comprise an available luer-activated connector device having a number of components therein. However, for purposes of illustration clarity, the connector device 921 is shown as a generic connector having a pathway through which a secondary fluid flows through from the fluid injection device 916 through the anti-backflow device 938 and ultimately into the primary flow body 920 for deliver to a patient.

More particularly, the primary flow body 920 comprises a sidewall 922 and opposing first and second openings 924a and 924b. A flow channel 925 extends between the first and second openings 924a and 924b. The primary flow body 920 can be coupled to respective primary flow line segments (e.g., 18e and 18f of FIGS. 1A and 1B, or 108c and 108d of FIG. 1C) via respective first and second openings 924a and 924b, such as by an adhesive. Thus, the unidirectional access port assembly 918 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The primary flow housing 920 can comprise an access wall 925 that extends outwardly and orthogonally from the side wall 922 to form generally a T-shape configuration. In another example, the primary flow housing 920 can comprise a Y-shape configuration. The anti-backflow device 938 comprises a flow housing 931 having a first end 939a coupled to the access wall 925 by any number of coupling means, such as by an adhesive, a press fit, a male/female luer lock, etc. The other end 939b of the flow housing 931 can be coupled to a connection end 933 of the connector device 921, such as by a male/female luer lock configuration as shown. Alternatively, the connector device 921 can be permanently bonded to the flow housing 931.

The connector device 921 can comprise an access port opening 934 that defines an access port 914 and that receives an injection end 935 of the fluid injection device 916 for delivery of a secondary fluid from a fluid chamber (not shown) of the fluid injection device 916 into the access port 914 via a secondary fluid path S9 of the fluid injection device 916. As noted above, the connector device 921 can comprise a variety of components supported therein, such as a split septum, a spring, a fluid injection conduit, such as provided with available connectors that are luer-activated when a fluid injection device or syringe is threadably coupled to the connector device 921.

The anti-backflow device 938 therefore facilitates fluid communication to a primary fluid path P8 (of an IV set) via the access port 914. More specifically, the anti-backflow device 938 can further comprise an elastomeric component 937 that fluidly separates the access port 914 from the flow channel 925. The elastomeric component 937 can comprise a one-way compliant opening 936 operable to prevent the primary fluid from passing through the one-way compliant opening 936 into the fluid chamber of the fluid injection device 916 while the fluid injection device 916 is removably coupled to the connector device 921. In this example, the anti-backflow device 938 can be similar to the “duck-bill” elastomeric check valve shown in FIG. 2A. Thus, in the closed position of FIG. 9, the one-way compliant opening 936 prevents or restricts the primary fluid from passing into the access port opening 934, because the elastomeric properties of the anti-backflow device 938 are such that the elastomeric component 937 is rigid enough to overcome the fluid pressure applied by the primary fluid in the primary fluid path P9, and because the compliant opening 936 is formed as a slit so that the primary fluid cannot pass through without sufficient fluid pressure. In the open position (e.g., see also FIG. 2B), the one-way compliant opening 936 permits or allows the secondary fluid to pass from the fluid injection device 916 through the access port opening 934 and into the primary fluid path P9, because the elastomeric component 937 is compliant enough to give way to the fluid pressure of the secondary fluid applied by the fluid injection device 916 on one side of the elastomeric component 937, such that the slit “opens up” and protrudes inwardly towards the primary fluid path P9 for mixing the secondary fluid with the primary fluid for delivery to a patient. In one example, the anti-backflow device 938 can be a planar disk in the shape of a cylinder, but it can take other suitable shapes and forms.

The anti-backflow device 938 can be supported by the flow housing 931 of the anti-backflow device 938 in a number of ways. For instance, the flow housing 931 can be comprised of two housings that sandwich the perimeter portion 940 of the compliant component 937 (similar to as shown in FIG. 2A). In the example shown in FIG. 9, the compliant component 937 can be supported by and attached to an annular recess 941 formed in the flow housing 931, such that the perimeter portion 940 of the compliant component 937 is fixed, and so that the central area of the compliant component 937 can flex or stretch to open the one-way compliant opening 936 to allow the secondary fluid to pass through.

In one example, the anti-backflow device 938 can be removably coupled to the primary flow housing 920 via a male/female luer lock interface or some other type of interface or coupling, so that the anti-backflow device 938 (and perhaps the connector device 921) can be replaced with another anti-backflow device, such as one having a ball type check valve similar or the same as the examples discussed herein.

Similarly as exemplified above in other examples, the unidirectional access port assembly 918 is operable to maintain continuous connection to the fluid injection device 916 to the connector device 921 over multiple successive dosages of the secondary fluid from the fluid injection device 916 into the primary fluid path P9 through the anti-backflow device 938.

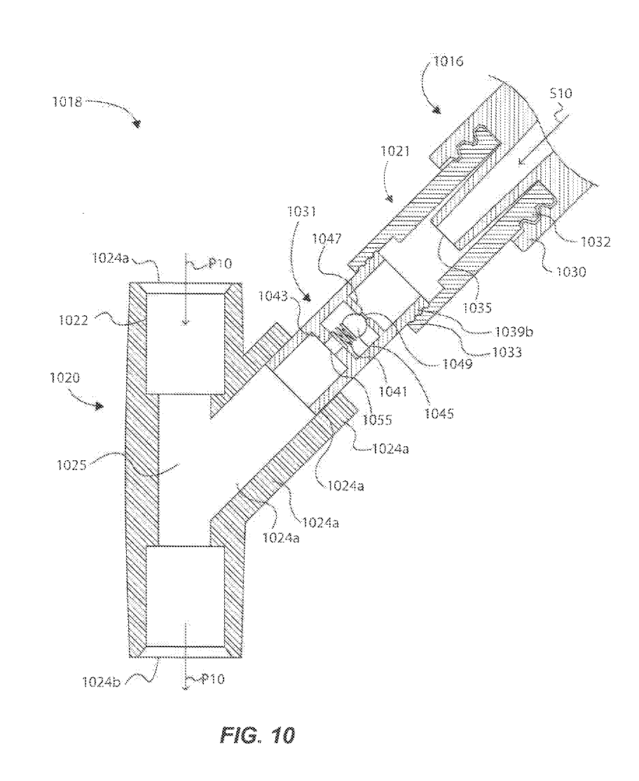

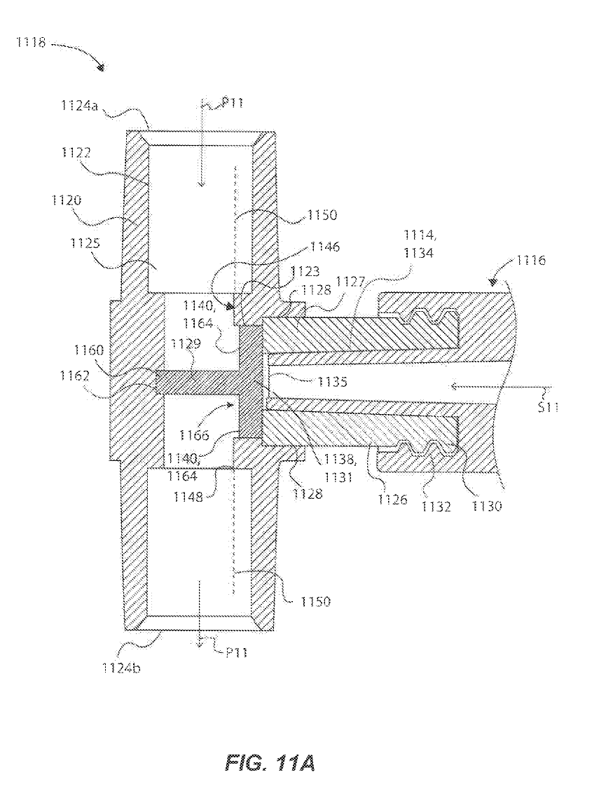

FIG. 10 illustrates an example unidirectional access port assembly 1018 in a closed position and coupled to a fluid injection device 1021. The unidirectional access port assembly 1018 can comprise three main components: a primary flow body 1020; an anti-backflow device 1038, and a connector device 1021. These three components can be coupled to each other in the manner shown such that the anti-backflow device 1038 is generally situated between the primary flow body 1020 and the connector device 1021. The fluid injection device 1016 can be removably coupled to the connector device 1021, such as via a male type connection portion 1030 interfaced with a female type connector portion 1032 of the fluid injection device 1016. In one example, the connector device 1021 can comprise an available luer-activated connector device having a number of components therein. However, for purposes of illustration clarity, the connector device 1021 is shown as a generic connector having a pathway through which a secondary fluid flows through from the fluid injection device 1016 through the anti-backflow device 1038 and ultimately to the primary flow body 1020 for deliver to a patient.

More particularly, the primary flow body 1020 comprises a sidewall 1022 and opposing first and second openings 1024a and 1024b. A flow channel 1025 extends between the first and second openings 1024a and 1024b. The primary flow body 1020 can be coupled to respective primary flow line segments (e.g., 18e and 18f of FIGS. 1A and 1B, or 108b and 108c of FIG. 1C) via respective first and second openings 1024a and 1024b, such as by an adhesive. Thus, the unidirectional access port assembly 1018 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The primary flow housing 1020 can comprise an access port body 1023 extending outwardly and transverse from the sidewall 1022 to form generally a Y-shape configuration (a T-shape configuration also being contemplated). The anti-backflow device 1038 comprises a flow housing 1031 having a first end 1039a coupled to the access port body 1023 by any number of coupling means, such as by an adhesive, a press fit, a male/female luer lock, etc. The other end 1039b of the flow housing 1031 can be coupled to a connection end 1033 of the connector device 1021, such as by a male/female luer lock configuration as shown. Alternatively, the connector device 1021 can be permanently bonded to the flow housing 1031. The flow housing 1031 could also be comprised of two bodies, such as the first and second access port bodies in FIG. 7, so that the components supported therein can be assembled appropriately inside the flow housing 1031.

The connector device 1021 can comprise an access port opening 1034 that defines an access port 1014 and that receives an injection end 1035 of the fluid injection device 1016 for delivery of a secondary fluid from a fluid chamber (not shown) of the fluid injection device 1016 into the access port 1014 via a secondary fluid path S10 of the fluid injection device 1016. As noted above, the connector device 1021 can comprise a variety of components supported therein, such as a split septum, a spring, a fluid conduit, such as with available connectors that are luer-activated when the fluid injection device 1016 is threadably coupled to the connector device 1021. The anti-backflow device 1038, therefore, facilitates fluid communication to a primary fluid path P10 (of an IV set) via the access port 1014.

In this example, the anti-backflow device 1038 can comprise a spring 1041 (e.g., a coil spring) seated at one end against a spring support portion 1043 of the primary flow body 1020. The spring support portion 1043 can define a secondary flow aperture 1055 through which the secondary fluid can flow from the fluid injection device 1016 when the anti-backflow device 1038 is opened. The spring 1041 may be seated by other means, such as in notches or protrusions of the spring support portion 1043 adjacent the end of the spring 1041 where seated. On the other end, the spring 1041 supports and seats a ball 1045. Specifically, in the closed position of FIG. 10, the spring 1041 biases the ball 1045 toward a ball support portion 1047 having a seat opening 1049 through which the secondary fluid passes when the anti-backflow device 1038 is opened by fluid pressure exerted secondary fluid injected by the fluid injection device 716.

Thus, in the closed position, the anti-backflow device 1038 prevents or restricts the primary fluid from passing into the access port opening 1034, because the spring 1041 is stiff enough to appropriately seat the ball 1045 against the ball support portion 1047 to close off the seat opening 1049 to prevent backflow of the primary fluid into the fluid injection device 1016. In the open position (not shown), the anti-backflow device 1038 permits or allows the secondary fluid to pass from the fluid injection device 1016 through the seat opening 1049 and into the primary fluid path P10, because the spring 1041 is compliant enough to give way to the fluid pressure of the secondary fluid applied by the fluid injection device 1016. Thus, fluid pressure applied via secondary fluid applies pressure to one side of the ball 1045 through the seat opening 1049, thereby causing compression of the spring 1041 while unseating the ball 1045 from the ball support portion 1047, thereby exposing the seat opening 1049 so that the secondary fluid can flow through the seat opening 1049 and around the ball 1045 and through the secondary flow aperture 1055 and into the primary fluid path P10. In one example, the anti-backflow device 1038 can be removably coupled to the primary flow housing 1020 via a male/female luer lock interface, so that the anti-backflow device 1038 can be replaced with another anti-backflow device, such as one having an elastomeric check valve similar or the same as the examples discussed herein.

Similarly as exemplified above in other examples, the unidirectional access port assembly 1018 is operable to maintain continuous connection to the fluid injection device 1016 to the connector device 1021 over multiple successive dosages of the secondary fluid from the fluid injection device 1016 into the primary fluid path P10 through the anti-backflow device 1038.

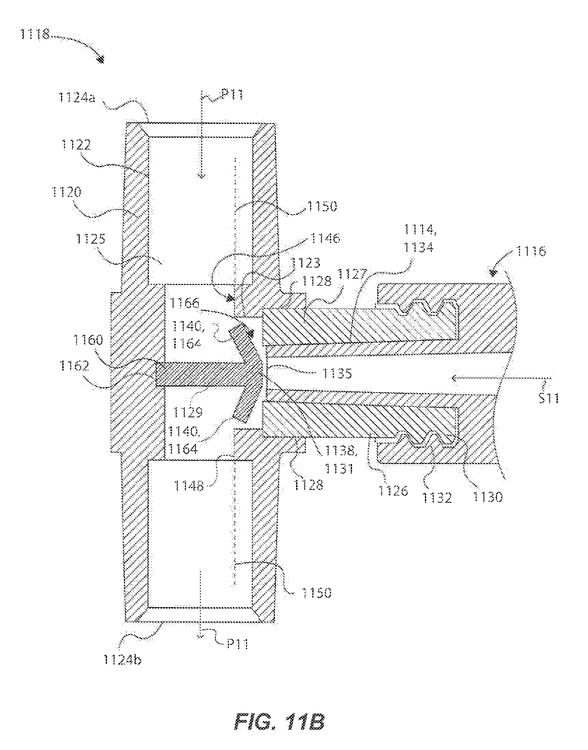

FIGS. 11A and 11B illustrate an example unidirectional access port assembly 1118 coupled to a fluid injection device 816, and showing the unidirectional access port assembly 1118 in a closed position (FIG. 11A) and an open position (FIG. 11B). The unidirectional access port assembly 1118 comprises a primary flow body 1120 comprising a sidewall 1122 and opposing first and second openings 1124a and 1124b. A flow channel 1125 extends between the first and second openings 1124a and 1124b. The primary flow body 1120 can be coupled to respective primary flow line segments (e.g., 18e and 18f of FIGS. 1A and 1B, or 108c and 108d of FIG. 1C) via respective first and second openings 1124a and 1124b, such as by an adhesive. Thus, the unidirectional access port assembly 1118 at least partially defines the flow line and the fluid path of an IV set or an IV set system, such as the primary IV set 15 (comprising the IV set 13 and the IV extension set 11, with the unidirectional access port assembly supported on the IV extension set 11) of FIG. 1A, or the IV set 106 of FIG. 1C.

The unidirectional access port assembly 1118 can comprise an access port body 1126 supported at least partially by the primary flow body 1120. The access port body 1126 extends outwardly and orthogonally from the sidewall 1122 to form generally a T-shape configuration. However, the port body 1126 can be configured to comprise other shapes, such as a Y-shape configuration. The access port body 1126 can be generally cylindrically shaped having one end attached (e.g., glued) to a side opening 1128 of the primary flow body 1120. On the other end, the access port body 1126 comprises a male type connection portion 1130 configured to removably couple a female type connector portion 1132 of a fluid injection device 1116. The access port body 1126 further comprises an access port opening 1134 that defines an access port 1114 and that receives an injection end 1135 of the fluid injection device 1116 for delivery of a secondary fluid from a fluid chamber (not shown) of the fluid injection device 1116 into the access port 1114 via a secondary fluid path S11 of the fluid injection device 1116. Note that the access port opening 1134 can be defined by a tapered sidewall that corresponds to the tapered shape of the injection end 1135 of the fluid injection device 1116 to define a tapered seal interface.

The unidirectional access port assembly 1118 can further comprise an anti-backflow device 1138 supported by the access port body 1126 and the primary flow body 1120, and that facilitates fluid communication to a primary fluid path P11 (of an IV set) via the access port 1114. More specifically, the anti-backflow device 1138 can comprise an elastomeric component that fluidly separates the access port 1114 from the flow channel 1125. As detailed below, the anti-backflow device 1138 is operable to prevent the primary fluid from passing from the flow channel 1125 and into a fluid chamber of the fluid injection device 1116 while the fluid injection device 1116 is removably coupled to the access port body 1126.

The anti-backflow device 1138 can comprise a valve support device 1129 and an elastic valve component 1140, which can be formed together as a unitary body formed of elastic material. In another example, the valve support device 1129 can be a separate component (e.g., made of rigid or semi-rigid material) attached to the left side of the elastic valve component 1140, such as via an adhesive. Each of these examples can be configured similarly as an umbrella type of valve.

The valve support device 1129 can be formed as a shaft or stem (so that fluid can pass around it via flow channel 1125), and the elastic valve component 1140 can be shaped as a cylindrically shaped disk (when in the closed position of FIG. 1 IA). A first (left) end 1160 of the valve support device 1129 can be supported by the primary flow body 1120, and in one example, the first end 1160 can be attached into a bore 1162. A second (right) end of the valve support device 1129 can support the elastic valve component 1140.

The elastic valve component 1140 can comprise a compliant perimeter portion 1164 that can be seated in a valve opening 1123 of the primary flow body 1120 to radially support the anti-backflow device 1138. The primary flow body 1120 can comprise a valve retention cavity 1166 that retains the elastic valve component 1140 when in the closed position (FIG. 11A). The valve retention cavity 1166 can be a three-dimensional area or volume defined by inner circular walls of the valve opening 1123 of the primary flow body 1120. Notably, when in the open position of FIG. 11B, the elastic valve component 1140 remains substantially contained within boundaries defined by the valve retention cavity 1166, because the compliant perimeter portion 1164, although deflected toward the flow channel 1125, does not extend inwardly beyond the valve retention cavity 1166. This is beneficial because it ensures that the elastic valve component 1140 does not deflect downwardly or compress onto itself due to fluid flow through the flow channel 1125.

The right face or side of the compliant perimeter portion 1164 is axially supported by an end portion 1127 of the access port body 1126, and a circular outer face of the compliant perimeter portion 1164 can be interfaced to and seated against the opening 1123 when in the closed position. In the open position, the secondary fluid is permitted to pass over and around the elastic valve component 1140. Once fluid pressure is removed via the fluid injection device 1116, the compliant perimeter portion 1164 automatically moves back to the closed position of FIG. 11A, due to the compliant, elastic nature of the elastic valve component 1140 being formed to be in a nominal state and configuration of FIG. 11A.

Notably, the injection end 1135 of the fluid injection device 1116 can be positioned very near one side of the elastic valve component 1140, such as 1 to 2 millimeters (as illustrated in FIG. 11A), to account for tolerances between the coupling of the fluid injection device 1116 to the access port body 1126. Thus, any possible “dead-space”is minimized. On the other side of the elastic valve component 1140 adjacent the primary fluid path P11, the anti-backflow device 1138 is positioned proximate or near the primary fluid path P11 to reduce or minimize dead-space on this side of the anti-backflow device 1138. More specifically, the sidewall 1122 can comprise an upper planar sidewall portion 1146 and a lower planar sidewall portion 1148 that define a flow plane 1150 along which the primary fluid generally flows through the flow channel 1125. When in the open position, the complaint perimeter portion 1140 of the anti-backflow device 1138 may slightly extend inwardly beyond this flow plane 1150, so that the secondary fluid can be injected and mixed with the primary fluid at a mixing region that is proximate the primary fluid path P11.

Similarly as exemplified above in other examples, the unidirectional access port assembly 1118 is operable to maintain continuous connection to the fluid injection device 1116 over multiple successive dosages of the secondary fluid from the fluid injection device 1116 into the primary fluid path P11 through the anti-backflow device 1138, because in response to removal of the fluid pressure of the secondary fluid via the injection end 1135, the elastic valve component 1140 automatically returns to the closed position to prevent backflow into the injection end 1135 (which may require removal of the injection end 1135 from the access port body 1126, and then swabbing of the access port body 1126 for sanitary purposes).

It is noted that the specific shape and configuration of the unidirectional access port assemblies discussed herein are not meant to be limiting in any way. Indeed, it is contemplated herein, for example, that any of the example unidirectional access port assemblies, and their corresponding port bodies shown in a Y-shape configuration could also comprise a T-shape configuration. This also applies to the unidirectional access port assemblies having a T-shape configuration, in that these could instead comprise a Y-shape configuration. As such, the specific shape of the unidirectional access ports are not meant to be limiting in any way, as will be appreciated by those skilled in the art.

It is noted herein that the unidirectional access port assemblies discussed herein can function to eliminate the need for a swabable connection access. Of course, however, if needed or desired, and as will be recognized by those skilled in the art, a swabable access port device or assembly can be connected directly to a unidirectional access port assembly so as to provide a swabable access port to which a fluid delivery device (e.g., a syringe) can be connected.

It is to be understood that the examples disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular examples only and is not intended to be limiting.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various examples of the present invention may be referred to herein along with alternatives for the various components thereof. It is understood that such examples and alternatives are not to be construed as de facto equivalents of one another, but are to be considered as separate and autonomous representations of the present invention.

Furthermore, the described features, structures, or characteristics may be combined in any suitable manner in one or more embodiments. In the description, numerous specific details are provided, such as examples of lengths, widths, shapes, etc., to provide a thorough understanding of the examples disclosed. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention.

1. A method for administering successive dosages of a medical fluid from a fluid injection device into an intravenous (IV) set, the method comprising:
obtaining an IV set having a primary flow line defining a primary fluid path, and a plurality of access points, at least one of the plurality of access points defining an access port;
establishing a fluid connection of a fluid source, having a primary fluid, with the primary fluid path of the IV set through one of the plurality of access points;
connecting a fluid injection device to a unidirectional access port assembly of the IV set to access the access port, the access port facilitating fluid communication between the fluid injection device and the primary fluid path through an anti-backflow device of the unidirectional access port assembly, the fluid injection device comprising a secondary fluid;
injecting a first dosage of the secondary fluid from the fluid injection device into the access port and the primary fluid flow path through the anti-backflow device;
maintaining the connection of the fluid injection device to the unidirectional access port assembly; and
injecting a second dosage of the secondary fluid from the fluid injection device into the access port and the primary fluid flow path through the anti-backflow device.

2. The method of claim 1, further comprising maintaining the connection of the fluid injection device to the unidirectional access port assembly, and injecting subsequent n number of additional dosages of the secondary fluid from the fluid injection device into the access port and the primary fluid flow path through the anti-backflow device.

3. The method of claim 1, wherein the anti-backflow device comprises a check valve operable to prevent the primary fluid from passing through the check valve into the fluid injection device between injection of the first dose and injection of the second dose, or n number of additional dosages.

4. The method of claim 1, wherein connecting the fluid injection device to the unidirectional access port assembly to access the access port comprises connecting a syringe to the unidirectional access port assembly by threadably engaging a female type connection portion of the syringe to a male type connection portion of the unidirectional access port assembly.

5. The method of claim 1, wherein injecting the first dosage of the secondary fluid from the fluid injection device into the access port and the primary fluid flow path through the anti-backflow device comprises injecting an anesthesia drug from a syringe.

6. The method of claim 1, wherein injecting the first dosage of the secondary fluid from the fluid injection device into the access port and the primary fluid flow path through the anti-backflow device comprises manually applying pressure to a plunger of the fluid injection device.

7. The method of claim 6, further comprising manually removing the application of pressure to the plunger of the fluid injection device prior to injecting the second dose, or subsequent n number of dosages.

8. The method of claim 1, furthering comprising disconnecting the fluid injection device from the unidirectional access port assembly, sterilizing the access port body, and connecting a subsequent fluid injection device, containing a secondary fluid, to the access port body.

9. The method of claim 1, wherein maintaining the connection of the fluid injection device to the unidirectional access port assembly comprises facilitating prevention of backflow of the primary fluid into the secondary fluid injection device via the anti-backflow device comprising a check valve, thereby preventing dilution of the secondary fluid with the primary fluid.

10. The method of claim 9, wherein the check valve comprises a one-way check valve comprising an elastomeric component having a slit operable between open and closed positions to facilitate or restrict fluid communication to the primary fluid path via the access port.

11. The method of claim 9, wherein the check valve comprises a one-way check valve comprising a valve support device and an elastic valve component, wherein a first end of the valve support device is supported by the primary flow body, and a second end of the valve support device supports the elastic valve component, the elastic valve component operable between open and closed positions to facilitate or restrict fluid communication to the primary fluid path via the access port.

12. A method for preventing dilution of a medical fluid in a fluid injection device coupled to an intravenous (IV) set during successive dosages with the fluid injection device, comprising:
obtaining an IV set having a primary flow line defining a primary fluid path, and a plurality of access points, at least one of the plurality of access points defining an access port;
establishing a fluid connection of a fluid source, having a primary fluid, with the primary fluid path of the IV set through one of the plurality of access points;
connecting a fluid injection device to a unidirectional access port assembly of the IV set to access the access port, the access port facilitating fluid communication between the fluid injection device and the primary fluid path through an anti-backflow device of the unidirectional access port assembly, the fluid injection device comprising a secondary fluid;
applying pressure to the fluid injection device to inject the secondary fluid from the fluid injection device into the primary fluid path through the anti-backflow device; and
removing pressure from the fluid injection device, while maintaining connection of the fluid injection device to the unidirectional access port assembly, in a manner such that the anti-backflow device restricts fluid flow from the primary fluid path to the fluid injection device, thereby preventing dilution of the secondary fluid in the fluid injection device.

13. The method of claim 12, wherein applying pressure to the fluid injection device to inject the secondary fluid from the fluid injection device into the primary fluid path through the anti-backflow device comprises injecting a first dosage of the secondary fluid from the fluid injection device into the access port and the primary fluid flow path through an anti-backflow device.

14. The method of claim 13, further comprising maintaining the connection of the fluid injection device to the unidirectional access port assembly after injecting the first dosage and after removing pressure from the fluid injection device.

15. The method of claim 14, further comprising applying pressure to the fluid injection device to inject a second dosage, or n number of additional dosages, of the secondary fluid from the fluid injection device into the access port and the primary fluid flow path through the anti-backflow device.

16. The method of claim 12, wherein the fluid injection device comprises a syringe removably coupled to the unidirectional access port assembly, and wherein the anti-backflow device comprises a check valve configured to prevent backflow of the primary fluid from the primary fluid path into a fluid chamber of the syringe, thereby preventing dilution of the secondary fluid in the fluid chamber.

17. A method for manufacturing a section of an intravenous (IV) set, comprising:
forming a primary flow body comprising a sidewall and opposing first and second openings, and a flow channel extending between the first and second openings;
forming an access port body supported at least partially by the primary flow body and that defines an access port operable to receive a fluid injection device;
supporting an anti-backflow device with at least one of the primary flow body or the access port body, wherein the primary flow body, the access port body, and the anti-backflow device form a unidirectional access port assembly; and
coupling the primary flow body of the unidirectional access port assembly to a first flow line segment, wherein the unidirectional access port assembly is operable, during use, to maintain continuous connection to the fluid injection device over multiple successive dosages of a secondary fluid from the fluid injection device into a primary fluid about a primary fluid path of an IV set through the anti-backflow device.

18. The method of claim 17, wherein the primary flow body and the access port body at least partially define a T-port type access port.

19. The method of claim 17, wherein the primary flow body and the access port body at least partially define a Y-site type access port.

20. The method of claim 17, wherein the anti-backflow device comprises an elastomeric component that fluidly separates an opening of the access port body and the flow channel primary flow body.

21. The method of claim 17, wherein the anti-backflow device comprises a ball type check valve.

22. The method of claim 17, wherein forming the access port body comprises forming a male type connector portion configured to removably couple a female type connector portion of the fluid injection device.

23. The method of claim 17, wherein the anti-backflow device comprises a one-way check valve comprising an elastomeric component having a slit that is operable between open and closed positions to facilitate or restrict fluid communication to the primary fluid path via the access port.

24. The method of claim 17, wherein the anti-backflow device comprises a one-way check valve comprising a valve support device and an elastic valve component, wherein a first end of the valve support device is supported by the primary flow body, and a second end of the valve support device supports the elastic valve component, the elastic valve component operable between open and closed positions to facilitate or restrict fluid communication to the primary fluid path via the access port.

25. The method of claim 17, further comprising forming an IV extension set operable to connect to the IV set, the method of forming the IV extension set comprising coupling a second flow line segment to the primary flow body of the unidirectional access port assembly opposite the first flow line segment, and coupling a first connector portion to an end of the first flow line segment and a second connector portion to an end of the second flow line segment, thereby defining an extension flow path of the IV extension set to further define the primary flow path, upon connection to the IV set.

26. An intravenous (IV) set, comprising:
a plurality of primary flow line segments defining, at least in part, a primary flow line and a primary fluid path, the IV set comprising a plurality of access points, at least one of the plurality of access points being an access port; and
a unidirectional access port assembly at least partially defining the primary flow line and the primary fluid path, the unidirectional access port assembly comprising:
a primary flow body comprising a sidewall and opposing first and second openings, and a flow channel extending between the first and second openings, the primary flow body being coupled to at least one of the primary flow line segments;
an access port body supported at least partially by the primary flow body and that defines an access port operable to receive a fluid injection device; and
an anti-backflow device supported at least partially by the access port body, and facilitating fluid communication to the primary fluid path via the access port,
wherein the unidirectional access port assembly is operable to maintain continuous connection to a fluid injection device over multiple successive dosages of a fluid from the fluid injection device into the primary fluid path through the anti-backflow device.

27. The IV set of claim 26, wherein the primary flow body and the access port body at least partially define a T-port type access port.

28. The IV set of claim 26, wherein the primary flow body and the access port body at least partially define a Y-site type access port.

29. The IV set of claim 26, wherein the anti-backflow device comprises an elastomeric component that fluidly separates the access port from the flow channel.

30. The IV set of claim 29, wherein the elastomeric component comprises a one-way compliant opening operable to prevent a primary fluid from passing through the one-way compliant opening into a fluid chamber of the fluid injection device while the fluid injection device is removably coupled to the access port body.

31. The IV set of claim 26, wherein the anti-backflow device comprises a valve support device and an elastic valve component, wherein a first end of the valve support device is supported by the primary flow body, and a second end of the valve support device supports the elastic valve component, the elastic valve component facilitating fluid communication to the primary fluid path via the access port.

32. The IV set of claim 31, wherein elastic valve component comprises a compliant perimeter portion operable to deflect to an open position in response to application of fluid pressure from the injection end via the access port, and operable to deflect to a closed position in response to removal of the fluid pressure.

33. The IV set of claim 32, wherein the elastic valve component is seated against the primary flow body and the access port body when the anti-backflow device is in a closed position.

34. The IV set of claim 32, wherein the access port body comprises a valve retention cavity that retains the elastic valve component when in the closed position, and wherein, when in the open position, the elastic valve component remains contained within boundaries defined by the valve retention cavity.

35. The IV set of claim 31, wherein the valve support device is formed as a cylindrically shaped stem attached to a bored of the primary flow body, and wherein the elastic valve component is formed as a circular disk.

36. The IV set of claim 26, wherein the anti-backflow device comprises an elastomeric component having a slit operable between open and closed positions to facilitate or restrict fluid communication to the primary fluid path via the access port.

37. The IV set of claim 36, wherein the elastomeric component comprises a duck-bill type one-way check valve.

38. The IV set of claim 37, wherein a circumferential perimeter portion of the elastomeric component is supported between the primary flow body and the access port body.

39. The IV set of claim 26, wherein the anti-backflow device comprises a ball type check valve operable to prevent a primary fluid from passing through the ball type check valve into a fluid chamber of the fluid injection device while the fluid injection device is removably coupled to the access port body.

40. The IV set of claim 26, wherein the access port body comprises a male type connection portion configured to removably couple a female type connector portion of the fluid injection device.

41. The IV set of claim 26, wherein the unidirectional access port assembly is coupled to adjacent primary flow line segments of the IV set at a location proximate a distal terminus of the IV set.

42. The IV set of claim 26, wherein one of the access points of the IV set is coupleable to a fluid source.

43. An IV set system, comprising:
an IV set comprising a plurality of primary flow line segments defining, at least in part, a primary flow line and a primary fluid path, the IV set comprising a plurality of access points, at least one of the plurality of access points being an access port;
an IV extension set connected to the IV set, the IV extension set comprising a first extension flow line segment having a first connector portion located about an end of the first extension flow line segment, and a second extension flow line segment having a second connector portion located at an end of the second extension flow line segment;
a unidirectional access port assembly supported on the IV extension set, the unidirectional access port assembly at least partially defining the primary flow line and the primary fluid path, and the unidirectional access port assembly comprising:
a primary flow body comprising a sidewall and opposing first and second openings, and a flow channel extending between the first and second openings, the primary flow body being coupled to at least one of the first or second extension flow line segments;
an access port body supported at least partially by the primary flow body and that defines an access port operable to receive a fluid injection device; and
an anti-backflow device associated with the access port, and facilitating fluid communication to the primary fluid path via the access port,
wherein the unidirectional access port assembly is operable to maintain continuous connection to a fluid injection device over multiple successive dosages of a fluid from the fluid injection device into the primary fluid path through the anti-backflow device.

44. An intravenous (IV) fluid delivery system, comprising:
a fluid source having a primary fluid;
an IV set comprising:
a plurality of primary flow line segments defining, at least in part, a primary flow line and a primary fluid path for transferring the primary fluid from the fluid source, the IV set comprising a plurality of access points, at least one of the plurality of access points being an access port;
a fluid injection device comprising a secondary fluid; and
a unidirectional access port assembly at least partially defining the primary flow line and the primary fluid path, the unidirectional access port assembly comprising:
a primary flow body comprising a sidewall and opposing first and second openings, and a flow channel extending between the first and second openings, the primary flow body being coupled to at least one of the primary flow line segments;
an access port body supported at least partially by the primary flow body and that defines an access port that receives the fluid injection device; and
an anti-backflow device supported at least partially by the unidirectional access port body, and facilitating fluid communication to the primary fluid path via the access port,
wherein the unidirectional access port assembly is operable to maintain continuous connection to the fluid injection device over multiple successive dosages of the secondary fluid from the fluid injection device into the primary fluid path through the anti-backflow device.

45. The fluid delivery system of claim 44, wherein the primary flow body and the access port body at least partially define one of a T-port type access port or a Y-site type access port.

46. The fluid delivery system of claim 44, wherein the anti-backflow device comprises one of an elastomeric component or a ball type check valve, the anti-backflow device operable to prevent a primary fluid from passing through the anti-backflow device into a fluid chamber of the fluid injection device while the fluid injection device is removably coupled to the access port body.

47. The fluid delivery system of claim 44, wherein the access port body comprises a male type connection portion configured to removably couple a female type connector portion of the fluid injection device.

48. The fluid delivery system of claim 44, wherein the anti-backflow device comprises an elastomeric component having a slit operable between open and closed positions to facilitate or restrict fluid communication to the primary fluid path via the access port.

49. The fluid delivery system of claim 44, wherein the anti-backflow device comprises a one-way check valve comprising a valve support device and an elastic valve component, wherein a first end of the valve support device is supported by the primary flow body, and a second end of the valve support device supports the elastic valve component, the elastic valve component operable between open and closed positions to facilitate or restrict fluid communication to the primary fluid path via the access port.

50. The fluid delivery system of claim 44, further comprising an IV extension set connected to the IV set, the IV extension set being in support of the unidirectional access port assembly.

51. A method for administering successive dosages, with the IV fluid delivery system of claim 44, of the secondary fluid from the fluid injection device to a patient during a medical procedure, the method comprising:
connecting the fluid injection device to the unidirectional access port assembly to access the access port, the access port facilitating fluid communication between the fluid injection device and the primary fluid path through an anti-backflow device of the unidirectional access port assembly, the fluid injection device comprising the secondary fluid;
injecting a first dosage of the secondary fluid from the fluid injection device into the access port and the primary fluid flow path through an anti-backflow device;
maintaining the connection of the fluid injection device to the unidirectional access port assembly; and
injecting a second dosage of the secondary fluid from the fluid injection device into the access port and the primary fluid flow path through the anti-backflow device.

52. An intravenous (IV) extension set for use with an IV set, the IV extension set comprising:
a plurality of extension flow line segments defining, at least in part, an extension flow line and an extension fluid path;
a first connector portion coupled to one of the extension flow line segments, the first connector portion configured to couple to a connector portion of an IV set;
a second connector portion coupled to another one of the extension flow line segments; and
a unidirectional access port assembly coupled to two of the plurality of extension flow line segments, the unidirectional access port assembly comprising:
a primary flow body comprising a sidewall and opposing first and second openings, and a flow channel extending between the first and second openings;
an access port body supported at least partially by the primary flow body that defines an access port operable to receive a fluid injection device; and
an anti-backflow device supported at least partially by the access port body, and facilitating fluid communication to the extension fluid path via the access port body,
wherein the unidirectional access port assembly is operable to maintain continuous connection to a fluid injection device over multiple successive dosages of a fluid from the fluid injection device into the primary fluid path through the anti-backflow device.

53. The IV extension set of claim 52, wherein the primary flow body and the access port body at least partially define a T-port type access port.

54. The IV extension set of claim 52, wherein the anti-backflow device comprises an elastomeric component that fluidly separates the access port from the flow channel.

55. The IV extension set of claim 54, wherein the elastomeric component comprises a one-way compliant opening operable to prevent a primary fluid from passing through the one-way compliant opening into a fluid chamber of the fluid injection device while the fluid injection device is removably coupled to the access port body.

56. The IV extension set of claim 52, wherein the anti-backflow device comprises a valve support device and an elastic valve component, wherein a first end of the valve support device is supported by the primary flow body, and a second end of the valve support device supports the elastic valve component, the elastic valve component facilitating fluid communication to the primary fluid path via the access port.

57. The IV extension set of claim 56, wherein elastic valve component comprises a compliant perimeter portion operable to deflect to an open position in response to application of fluid pressure from the injection end via the access port body, and operable to deflect to a closed position in response to removal of the fluid pressure.

58. The IV extension set of claim 52, wherein the anti-backflow device comprises an elastomeric component having a slit operable between open and closed positions to facilitate or restrict fluid communication to the primary fluid path via the access port body.

59. The IV extension set of claim 58, wherein the elastomeric component comprises a duck-bill one-way check valve.
